SEQUENCE LISTING (13 sequences)

SEQ ID NO: 1 - protein, 525 aa, Homo sapiens
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 2 - protein, 218 aa, Artificial Sequence
  Ab6 light chain
DIVMTQTPLSLSVTPGQPASISCKASQSLDYEGDSDMNWYLQKPGQPPQLLIYGASNLESGVPDRFSGSGSGTDFTLKISRVEAEDVGVYYCQQSTEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 446 aa, Artificial Sequence
  Ab6 heavy chain
QMQLVQSGPEVKKPGTSVKVSCKASGYTFTDYNVDWVRQARGQRLEWIGDINPNDGGTIYAQKFQERVTITVDKSTSTAYMELSSLRSEDTAVYYCARNYRWFGAMDHWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 111 aa, Artificial Sequence
  Ab6 light chain immunoglobulin variable domain
DIVMTQTPLSLSVTPGQPASISCKASQSLDYEGDSDMNWYLQKPGQPPQLLIYGASNLESGVPDRFSGSGSGTDFTLKISRVEAEDVGVYYCQQSTEDPRTFGGGTKVEIK

SEQ ID NO: 5 - protein, 119 aa, Artificial Sequence
  Ab6 heavy chain immunoglobulin variable domain
QMQLVQSGPEVKKPGTSVKVSCKASGYTFTDYNVDWVRQARGQRLEWIGDINPNDGGTIYAQKFQERVTITVDKSTSTAYMELSSLRSEDTAVYYCARNYRWFGAMDHWGQGTTVTVSS

SEQ ID NO: 6 - protein, 15 aa, Artificial Sequence
  Ab6 CDR-L1
KASQSLDYEGDSDMN

SEQ ID NO: 7 - protein, 7 aa, Artificial Sequence
  Ab6 CDR-L2
GASNLES

SEQ ID NO: 8 - protein, 9 aa, Artificial Sequence
  Ab6 CDR-L3
QQSTEDPRT

SEQ ID NO: 9 - protein, 5 aa, Artificial Sequence
  Ab6 CDR-H1
DYNVD

SEQ ID NO: 10 - protein, 17 aa, Artificial Sequence
  Ab6 CDR-H2
DINPNDGGTIYAQKFQE

SEQ ID NO: 11 - protein, 10 aa, Artificial Sequence
  Ab6 CDR-H3
NYRWFGAMDH

SEQ ID NO: 12 - protein, 7 aa, Artificial Sequence
  PLBL2 peptide
LTFPTGR

SEQ ID NO: 13 - protein, 8 aa, Artificial Sequence
  LPLA2 peptide
IPVIGPLK

(19) United States
(12) Patent Application Publication
(10) Pub. No.: US 2023/0077205 A1
(43) Pub. Date: Mar. 9, 2023

(54) METHODS OF SEPARATING HOST CELL LIPASES FROM AN ANTI-LAG3 ANTIBODY PRODUCTION

(71) Applicant: Merck Sharp & Dohme LLC, Rahway, NJ (US)

(72) Inventors: Colette M. CUTLER, Bloomingdale, NJ (US); Hong LI, Basking Ridge, NJ (US); Sketa PATEL, Parsippany, NJ (US); Sandra E. RIOS, Berkely Heights, NJ (US); John P. WELSH, Charlottesville, VA (US)

(73) Assignee: Merck Sharp & Dohme LLC, Rahway (US)

(21) Appl. No.: 17/796,023

(22) PCT Filed: Jan. 28, 2021
(86) PCT No.: PCT/US2021/015368

§ 371 Date: Jul. 28, 2022

Related U.S. Application Data
(60) Provisional application No. 62/967,347, filed on Jan. 29, 2020.

(51) Int. Cl. C07K 16/06; C07K 16/28; C12N 9/20; A61K 39/395; A61K 47/26; A61K 47/22; A61K 47/18; A61K 47/20; B01D 15/32; B01D 15/42; B01D 15/36

(52) U.S. Cl. CPC C07K 16/065; C07K 16/2803; C12Y 301/01004; C12N 9/20; A61K 39/39591; A61K 47/26; A61K 47/22; A61K 47/183; A61K 47/20; B01D 15/327; B01D 15/424; B01D 15/362; C07K 2317/565; C07K 2317/567

(57) ABSTRACT

Provided herein are methods of separating host cell lipases from an anti-LAG3 antibody or antigen binding fragment in chromatographic processes and methods of improving polysorbate-80 stability in an anti-LAG3 antibody formulation by separating host cell lipases from the anti-LAG3 antibody or antigen binding fragment using chromatographic processes. Also provided are pharmaceutical compositions comprising an anti-LAG3 antibody or antigen binding fragment and less than 2 ppm of a host cell lipase.

### CROSS-REFERENCE TO RELATED APPLICATIONS

This Application claims the benefit of U.S. Provisional Patent Application No. 62/967,347, filed Jan. 29, 2020, which is herein incorporated by reference in its entirety.

### REFERENCE TO SEQUENCE LISTING SUBMITTED ELECTRONICALLY

This application contains a Sequence Listing which has been submitted electronically in ASCII format and is hereby incorporated by reference in its entirety. Said ASCII copy, created on Jan. 19, 2021, is named 24955WOPCT-SEQTXT-19JAN2021.txt and is 14.2 kilobytes in size.

### FIELD OF THE INVENTION

Provided herein are methods of separating host cell proteins (HCP) (e.g., lipases) from an anti-LAG3 antibody in chromatographic processes. Also provided herein are methods of improving polysorbate-80 (PS-80) stability in an anti-LAG3 antibody formulation (e.g., drug substance formulation or drug product formulation) by separating HCP (e.g., lipases) from the anti-LAG3 antibody (e.g., monoclonal antibody) using chromatographic processes.

### BACKGROUND OF THE INVENTION

LAG-3 (Lymphocyte Activation Gene-3) is a cell surface molecule expressed on activated T cells, B cells, NK cells, and plasmacytoid dendritic cells. LAG-3 is structurally similar to CD4, and binds to MHC class II molecules as an inhibitory receptor. LAG-3 was shown to negatively regulate T-cell activation and proliferation, as well as to be co-expressed on tumor-infiltrating lymphocytes with other inhibitory receptors. Expression of LAG3 is indicative of a highly exhausted T-cell phenotype. See Goldberg MV1, Drake C G. Curr. Top. Microbiol. Immunol. 2011; 344:269-78.

In bioprocessing and manufacturing of antibodies (e.g., monoclonal antibodies), host cell proteins (HCP) (e.g., lipases) constitute part of the impurities that are often difficult to remove from the antibodies. Such impurities can cause various issues in the safety and efficacy of biopharmaceuticals. Regulatory agencies throughout the world require that biopharmaceutical products meet certain acceptance criteria, including the level of impurities and tests for detection and quantification of impurities. Several anti-LAG3 antibodies are in clinical development, and it is desirable to develop efficient and effective processes to remove HCP (e.g., lipases) from these antibodies.

### SUMMARY OF THE INVENTION

The present disclosure provides methods of separating HCP (e.g., lipases) from an anti-LAG3 antibody or antigen-binding fragment through chromatographic processes as well as methods of improving PS-80 stability in an anti-LAG3 antibody formulation (e.g., drug substance formulation or drug product formulation) by separating HCP (e.g., lipases) from an anti-LAG3 antibody or antigen-binding fragment using Hydrophobic Interaction (HIC) or Cation Exchange (CEX) chromatographic processes. The disclosure is based, at least in part, on the discovery that the HCP (e.g., lipases) and the anti-LAG3 antibody or antigen binding fragment can be sufficiently separated under operating conditions where the separation factor (α) between the two proteins and/or the partition coefficient (Kₚ) for the HCP (e.g., lipase) reach certain ranges of numeric values.

In one embodiment, the lipase is PLBL2. In yet another embodiment, the lipase is LPLA2. In one embodiment, the lipase is LP-PLA2. In one embodiment, the HCP is Clusterin.

In another aspect, provided herein is a pharmaceutical composition comprising the anti-LAG3 antibody or antigen-binding fragment and less than 2 ppm of a host cell lipase. The disclosure also provides a pharmaceutical composition comprising an anti-LAG3 antibody or antigen-binding fragment and polysorbate 80 (PS80) or polysorbate 20 (PS20) when formulated, wherein at 3 months at 2-8° C., the concentration of PS80 or PS20 is maintained at ≥90% of the concentration when formulated.

### BRIEF DESCRIPTION OF THE DRAWINGS

FIG. 1 shows PLBL2 or LPLA2 log K_{P} values for a range of HIC conditions typical for modulation of binding by salt concentration.

FIG. 2 shows a comparison of log K_{P} values on a HIC resin for PLBL2, LPLA2, and two different mAbs, mAb2 (Ab6) and mAb3. mAb3 has very similar binding to HIC when compared to PLBL2 and LPLA2, but mAb2 is bound much more weakly than mAb3, PLBL2, and LPLA2, offering greater separation potential of PLBL2 and LPLA2 from mAb2 than from mAb3.

FIG. 3 show PS-80 concentration of the Ab6 AEX pool drug substance (AEX DS), and Ab6 HIC bind and elute pool drug substance (HIC B&E DS) or Ab6 HIC flowthrough drug substance (HIC FT DS) at 5±3° C. at 2, 4, 6 and 14 week intervals.

FIG. 4 shows PS-80 concentration of the Ab6A drug product of Example 6 at 5° C.±3° C. (inverted), at the accelerated condition of 25° C. (25° C.±2° C., 60% relative humidity, inverted), and at the stressed condition of 40° C. (40° C.±2° C., 75% relative humidity, inverted) at 3 months.

### DETAILED DESCRIPTION OF THE INVENTION

### Definitions

Certain technical and scientific terms are specifically defined below. Unless specifically defined elsewhere in this document, all other technical and scientific terms used herein have the meaning commonly understood by one of ordinary skill in the art to which this disclosure relates. In case of conflict, the present specification, including definitions, will control.

The term “operating condition,” “operation condition,” “processing condition,” or “process condition,” as used exchangeably herein, refers to the condition for operating a chromatographic process. The operating condition can be equilibration condition, loading condition, wash condition, and/or elution condition, etc. The operating condition includes but is not limited to the type of the chromatographic resin, the resin backbone, the resin ligand, the pH of the operating solution, the composition of the operating solution, the concentration of each ingredient of the operating solution, the conductivity of the operating solution, the ionic strength of the operating solution, the cationic strength of the operating solution, the anionic strength of the operating solution, or a combination of two or more above factors.

The term “operating solution” refers to the solution used in operating a chromatographic process. The operating solution can be equilibration solution, loading or feed solution, wash solution, and/or elution solution, etc.

The term “partition coefficient” or “Kₚ,” as used herein, refers to the ratio of the concentration of a protein bound to a chromatographic resin (Q) to the concentration of the protein remaining in the solution (C) at equilibrium under a specific operating condition. The partition coefficient for a particular protein can be calculated as follows: Kₚ=Q/C.

The term “separation factor” or “α,” as used herein, refers to the ratio of the partition coefficient for a first protein (K_{p, protein 1}) and the partition coefficient for a second protein (K_{p, protein 2}). The separation factor quantifies the selectivity of a chromatographic resin between the two proteins, under a specific operating condition. It can be used to predict the extent of separation of the two proteins through the chromatographic resin under the operating condition. The separation factor between two proteins can be calculated as follows: α=K_{p, protein 1}/K_{p, protein 2}; or log α=log K_{p, protein 1}−log K_{p, protein 2}.

“Eluate,” as used herein, refers to the liquid that passes through a chromatography. In some embodiments, the eluate is the flowthrough of a loading solution. In other embodiments, the eluate comprises the elution solution that passes through the chromatography and any additional components eluted from the chromatography.

“Polysorbate-80 stability” or “PS-80 stability,” as used herein, refers to the state of PS-80 remaining physically, chemically, and/or biologically stable under common storage conditions (e.g., 5° C.±3° C., 25° C.±3° C., 60%±5% relative humidity (RH), 40° C.±2° C., 75%±5% relative humidity (RH)) over a period of time (e.g., 1 week, 1 month, 6 months, 1 year, 2 years, etc.). The PS-80 stability can be measured by the amount of intact PS-80 molecules and/or the amount of degraded products using various methods, including but not limited to mass spectrometry (MS), liquid chromatography-mass spectrometry (LCMS), liquid chromatography-multiple reaction monitoring (LC-MRM-MS) or solid phase extraction (SPE) on a HPLC system with a charged aerosol detector (CAD).

The term “about”, when modifying the quantity (e.g., mM, or M) of a substance or composition, the percentage (v/v or w/v) of a formulation component, the pH of a solution/formulation, or the value of a parameter characterizing a step in a method, or the like refers to variation in the numerical quantity that can occur, for example, through typical measuring, handling and sampling procedures involved in the preparation, characterization and/or use of the substance or composition; through instrumental error in these procedures; through differences in the manufacture, source, or purity of the ingredients employed to make or use the compositions or carry out the procedures; and the like. In certain embodiments, “about” can mean a variation of ±0.1%, 0.5%, 1%, 2%, 3%, 4%, 5%, or 10% of the value.

The phrase “maintained at ≥80, 85, 90, 95, or 99% of the concentration when formulated” when used in the context of measuring PS80 or PS20 stability after a period of time takes into consideration assay variability of ±10% in measurement of the PS80 or PS20 concentration.

As used herein, an “Ab6 variant” means a monoclonal antibody which comprises heavy chain and light chain sequences that are substantially identical to those in antibody Ab6 (as described below and in WO2016028672, incorporated by reference in its entirety), except for having three, two or one conservative amino acid substitutions at positions that are located outside of the light chain CDRs and six, five, four, three, two or one conservative amino acid substitutions that are located outside of the heavy chain CDRs, e.g., the variant positions are located in the FR regions or the constant region of the immunoglobulin chain(s), and optionally has a deletion of the C-terminal lysine residue of the heavy chain. In other words, Ab6 and a Ab6 variant comprise identical CDR sequences, but differ from each other due to having a conservative amino acid substitution at no more than three or six other amino acid positions in the full length light and heavy chain sequences, respectively. An Ab6 variant is substantially the same as Ab6 with respect to the following properties: binding affinity to human LAG3 and ability to block the binding of human LAG3 to human MHC Class II.

As used herein, the term “antibody” refers to any form of antibody that exhibits the desired biological or binding activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, chimeric antibodies and camelized single domain antibodies. “Parental antibodies” are antibodies obtained by exposure of an immune system to an antigen prior to modification of the antibodies for an intended use, such as humanization of an antibody for use as a human therapeutic.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

The variable regions of each light/heavy chain pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, Md.; 5^{th} ed.; NUT Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

As used herein, unless otherwise indicated, “antibody fragment” or “antigen binding fragment” refers to antigen binding fragments of antibodies, i.e. antibody fragments that retain the ability to bind specifically to the antigen bound by the full-length antibody, e.g. fragments that retain one or more CDR regions. Examples of antibody binding fragments include, but are not limited to, Fab, Fab′, F(ab′)₂, and Fv fragments; diabodies; linear antibodies; single-chain antibody molecules, e.g., sc-Fv; nanobodies and multispecific antibodies formed from antibody fragments.

“Chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

“Human antibody” refers to an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

“Comprising” or variations such as “comprise”, “comprises” or “comprised of” are used throughout the specification and claims in an inclusive sense, i.e., to specify the presence of the stated features but not to preclude the presence or addition of further features that may materially enhance the operation or utility of any of the embodiments of the invention, unless the context requires otherwise due to express language or necessary implication.

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 1 below.

TABLE 1
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

“Consists essentially of,” and variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition. As a non-limiting example, an anti-LAG3 antibody or antigen binding fragment that consists essentially of a recited amino acid sequence may also include one or more amino acids, including substitutions of one or more amino acid residues, which do not materially affect the properties of the binding compound.

“Framework region” or “FR” as used herein means the immunoglobulin variable regions excluding the CDR regions.

“Kabat” as used herein means an immunoglobulin alignment and numbering system pioneered by Elvin A. Kabat ((1991) Sequences of Proteins of Immunological Interest, 5th Ed. Public Health Service, National Institutes of Health, Bethesda, Md.).

Human LAG3 comprises the amino acid sequence:

(SEQ ID NO: 1)

MWEAQFLGLL FLQPLWVAPV KPLQPGAEVP VVWAQEGAPA
QLPCSPTIPL QDLSLLRRAG VTWQHQPDSG PPAAAPGHPL
APGPHPAAPS SWGPRPRRYT VLSVGPGGLR SGRLPLQPRV
QLDERGRQRG DFSLWLRPAR RADAGEYRAA VHLRDRALSC
RLRLRLGQAS MTASPPGSLR ASDWVILNCS FSRPDRPASV
HWFRNRGQGR VPVRESPHHH LAESFLFLPQ VSPMDSGPWG
CILTYRDGFN VSIMYNLTVL GLEPPTPLTV YAGAGSRVGL
PCRLPAGVGT RSFLTAKWTP PGGGPDLLVT GDNGDFTLRL
EDVSQAQAGT YTCHIHLQEQ QLNATVTLAI ITVTPKSFGS
PGSLGKLLCE VTPVSGQERF VWSSLDTPSQ RSFSGPWLEA
QEAQLLSQPW QCQLYQGERL LGAAVYFTEL SSPGAQRSGR
APGALPAGHL LLFLILGVLS LLLLVTGAFG FHLWRRQWRP
RRFSALEQGI HPPQAQSKIE ELEQEPEPEP EPEPEPEPEP
EPEQL;

see also Uniprot accession no. P18627. Residues 1-22 are the native leader sequence.

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

As used herein, including the appended claims, the singular forms of words such as “a,” “an,” and “the,” include their corresponding plural references unless the context clearly dictates otherwise. Unless otherwise required by context, singular terms shall include pluralities and plural terms shall include the singular.

As used herein, the terms “at least one” item or “one or more” item each include a single item selected from the list as well as mixtures of two or more items selected from the list.

Any example(s) following the term “e.g.” or “for example” is not meant to be exhaustive or limiting.

Unless expressly stated to the contrary, all ranges cited herein are inclusive; i.e., the range includes the values for the upper and lower limits of the range as well as all values in between. As an example, temperature ranges, percentages, ranges of equivalents, and the like described herein include the upper and lower limits of the range and any value in the continuum there between. All ranges also are intended to include all included sub-ranges, although not necessarily explicitly set forth. For example, a range of pH 4.0-5.0 is intended to include pH 4.0, 4.1, 4.13, 4.2, 4.1-4.6, 4.3-4.4, and 5.0. In addition, the term “or,” as used herein, denotes alternatives that may, where appropriate, be combined; that is, the term “or” includes each listed alternative separately as well as their combination.

Where aspects or embodiments of the disclosure are described in terms of a Markush group or other grouping of alternatives, the present disclosure encompasses not only the entire group listed as a whole, but each member of the group individually and all possible subgroups of the main group, but also the main group absent one or more of the group members. The present disclosure also envisages the explicit exclusion of one or more of any of the group members in the claims.

Exemplary methods and materials are described herein, although methods and materials similar or equivalent to those described herein can also be used in the practice or testing of the present disclosure. The materials, methods, and examples are illustrative only and not intended to be limiting.

### Anti-LAG3 Antibody

In one embodiment, the anti-LAG3 antibody is Ab6 or an Ab6 variant.

Ab6 has the following antibody components:

a light chain immunoglobulin with the amino acid sequence:

(SEQ ID NO: 2)
DIVMTQTPLSLSVTPGQPASISCKASQSLDYEGDSDMNWYLQKPGQPPQL
LIYGASNLESGVPDRFSGSGSGTDFTLKISRVEAEDVGVYYCQQSTEDPR
TFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC;

a heavy chain immunoglobulin with the amino acid sequence:

(SEQ ID NO: 3)
QMQLVQSGPEVKKPGTSVKVSCKASGYTFTDYNVDWVRQARGQRLEWIGD
INPNDGGTIYAQKFQERVTITVDKSTSTAYMELSSLRSEDTAVYYCARNY
RWFGAMDHWGQGTTVTVSSASTKGPSVFPLAPCSRSTSESTAALGCLVKD
YFPEPVTVSWNSGALTSGVHTFPAVLQSSGLYSLSSVVTVPSSSLGTKTY
TCNVDHKPSNTKVDKRVESKYGPPCPPCPAPEFLGGPSVFLFPPKPKDTL
MISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGK;

a light chain immunoglobulin variable domain with the amino acid sequence:

(SEQ ID NO: 4)
DIVMTQTPLSLSVTPGQPASISCKASQSLDYEGDSDMNWYLQKPGQPPQL
LIYGASNLESGVPDRFSGSGSGTDFTLKISRVEAEDVGVYYCQQSTEDPR
TFGGGTKVEIK;

a heavy chain immunoglobulin variable domain with the amino acid sequence:

(SEQ ID NO: 5)
QMQLVQSGPEVKKPGTSVKVSCKASGYTFTDYNVDWVRQARGQRLEWIGD
INPNDGGTIYAQKFQERVTITVDKSTSTAYMELSSLRSEDTAVYYCARNY
RWFGAMDHWGQGTTVTVSS;

and the following CDRs:

| | CDR-L1: |
|---|---|
| (SEQ ID NO: 6) | |
| | KASQSLDYEGDSDMN; |
| | CDR-L2: |
| (SEQ ID NO: 7) | |
| | GASNLES; |
| | CDR-L3: |
| (SEQ ID NO: 8) | |
| | QQSTEDPRT; |
| | CDR-H1: |
| (SEQ ID NO: 9) | |
| | DYNVD; |
| | CDR-H2: |
| (SEQ ID NO: 10) | |
| | DINPNDGGTIYAQKFQE; |
| | and |
| | CDR-H3: |
| (SEQ ID NO: 11) | |
| | NYRWFGAMDH |

In some preferred embodiments of the method of the present invention, the anti-LAG3 antibody, or antigen binding fragment thereof comprises: (a) light chain CDRs SEQ ID NOs: 6, 7 and 8, and (b) heavy chain CDRs SEQ ID NOs: 9, 10 and 11.

In other preferred embodiments of the method of the present invention, the anti-LAG3 antibody, or antigen binding fragment thereof comprises (a) a heavy chain variable region comprising SEQ ID NO:5, and (b) a light chain variable region comprising SEQ ID NO:4. In another preferred embodiment of the method of the present invention, the anti-LAG3 antibody comprises (a) a heavy chain comprising SEQ ID NO: 3 and (b) a light chain comprising SEQ ID NO:2. In another preferred embodiment of the method of the present invention, the anti-LAG3 antibody has two heavy chains and two light chains, wherein (a) the heavy chain consists of SEQ ID NO: 3 and (b) the light chain consists of SEQ ID NO:2.

In one embodiment, the anti-LAG3 antibody or antigen-binding fragment comprises a heavy chain constant region, e.g. a human constant region, such as γ1, γ2, γ3, or γ4 human heavy chain constant region or a variant thereof. In another embodiment, the anti-LAG3 antibody or antigen-binding fragment comprises a light chain constant region, e.g. a human light chain constant region, such as lambda or kappa human light chain region or variant thereof. By way of example, and not limitation, the human heavy chain constant region can be γ4 and the human light chain constant region can be kappa. In an alternative embodiment, the Fc region of the antibody is γ4 with a Ser228Pro mutation (Schuurman, J et. al., Mol. Immunol. 38: 1-8, 2001).

In some embodiments, different constant domains may be appended to humanized V_{L} and V_{H} regions derived from the CDRs provided herein. For example, if a particular intended use of an antibody (or fragment) of the present invention were to call for altered effector functions, a heavy chain constant domain other than human IgG1 may be used, or a hybrid IgG1/IgG4 may be utilized.

### Chromatographic Processes

The chromatographic process for the separation of host cell lipase from the anti-LAG3 antibody or antigen binding fragment can be a CEX chromatographic process. In another embodiment, the chromatographic process is a HIC chromatographic process. The foregoing chromatographic processes can be proceeded or followed by one or more of a CEX, AEX, mixed mode IEX, mixed mode AEX, mixed mode CEX, affinity chromatographic process, protein A or protein G affinity chromatographic process, immobilized metal affinity chromatographic (IMAC) process, and HAC chromatographic process. In one embodiment, the CEX or HIC chromatographic process is preceded by a protein A chromatography followed by AEX chromatography. In one embodiment, the CEX or HIC chromatographic process is preceded by a protein A chromatography performed in bind and elute mode followed by AEX chromatography performed in flowthrough mode.

IEX chromatography separates molecules based on net charge of the molecules. Separation occurs as a result of competition between the charged molecule of interest and counter ions for oppositely charged ligand groups on the IEX chromatographic resin. Strength of the binding of the molecule to the IEX resin depends on the net charge of the molecules, which is affected by operating conditions, such as pH and ionic strength. IEX resins include AEX resins and CEX resins. AEX resins may contain substituents such as diethylaminoethyl (DEAE), trimethyalaminoethyl (TMAE), quaternary aminoethyl (QAE) and quaternary amine (O) groups. CEX resins may contain substituents such as carboxymethyl (CM), sulfoethyl (SE), sulfopropyl (SP), phosphate (P) and sulfonate (S). Cellulosic IEX resins such as DE23, DE32, DE52, CM-23, CM-32 and CM-52 are available from Whatman Ltd. Maidstone, Kent, U.K. Sephadex-based and cross-linked IEX resins are also known. For example, DEAE-, QAE-, CM-, and SP-Sephadex, and DEAE-, Q-, CM- and S-Sepharose, and Sepharose are all available from GE Healthcare, Piscataway, N.J. Further, both DEAE and CM derived ethylene glycol-methacrylate copolymer such as TOYOPEARL™ DEAE-650S or M and TOYOPEARL™ CM-650S or M are available from Toso Haas Co., Philadelphia, Pa. POROS™ HS, POROS™ HQ, POROS™ XS are available from Thermo Fisher Scientific, Waltham, Mass.

HIC chromatography separates molecules based on hydrophobicity of molecules. Hydrophobic regions in the molecule of interest bind to the HIC resin through hydrophobic interaction. Strength of the interaction depends on operating conditions such as pH, ionic strength, and salt concentration. In general, HIC resins contain a base matrix (e.g., cross-linked agarose or synthetic copolymer material) to which hydrophobic ligands (e.g., alkyl or aryl groups) are coupled. Non-limiting examples of HIC resins include Phenyl SEPHAROSE™ 6 FAST FLOW™ (Pharmacia LKB Biotechnology, AB, Sweden); Phenyl SEPHAROSET™ High Performance (Pharmacia LKB Biotechnology, AB, Sweden); Octyl SEPHAROSET™ High Performance (Pharmacia LKB Biotechnology, AB, Sweden); Fractogel™ EMID Propyl or FRACTOGEL™ EMD Phenyl (E. Merck, Germany); MACRO-PREP™ Methyl or MACRO-PREP™ t-Butyl Supports (Bio-Rad, CA); WP HI-Propyl (C₃)™ (J. T. Baker, NJ); TOYOPEARL™ ether, phenyl or butyl (TosoHaas, PA); and Tosoh-Butyl-650M (Tosoh Corp., Tokyo, Japan).

HAC chromatography uses an insoluble hydroxylated calcium phosphate of the formula [Ca₁₀(PO₄)₆(OH)₂] as both the matrix and the ligand. The functional groups of the HAC resin include pairs of positively charged calcium ions (C-sites) and negatively charged phosphate groups (P-sites). The C-sites can interact with carboxylate residues on the protein surface while the P-sites can interact with basic protein residues. Strength of the binding between the protein and the HAC resin depends on operating conditions including pH, ionic strength, composition of solution, concentration of each component of the composition, gradient of pH, gradient of component concentration, etc. Various HAC resins, such as CHT™ Ceramic Hydroxyapatite and CFT™ Ceramic Fluoroapatite, are commercially available.

Affinity chromatography separates molecules based on a highly specific interaction between the molecule of interest and the functional group of the resin, such as interaction between antigen and antibody, enzyme and substrate, receptor and ligand, or protein and nucleic acid, etc. Some commonly used affinity chromatographic resins include protein A or protein G resin to purify antibodies, avidin biotin resin to purify biotin/avidin and their derivatives, glutathione resin to purify GST-tagged recombinant proteins, heparin resin to separate plasma coagulation proteins, IMAC resin to purify proteins that specifically interact with the metal ions, etc. Operating conditions of each affinity chromatography depend on the mechanism of the interaction and factors that affect the interaction. Commercial affinity chromatographic resins include but are not limited to MabSelect Sure, UNOsphere SUPrAh, Affi-Gel®, and Affi-Prep®.

The mixed mode can be a combination of any two or more functions or mechanisms described above or understood by a person of ordinary skill in the art, such as a combination of IEX and HIC (e.g., AEX/HIC or CEX/HIC), a combination of AEX and CEX (AEX/CEX), or a combination of HIC, AEX, and CEX (HIC/AEX/CEX), etc. Exemplary mixed mode chromatographic resins include but are not limited to OminPac PCX-500, Primesep®, Obelisc R, Oblisc N, Acclaim Trinity P1, Acclaim Trinity P2, Capto Adhere, Capto Adhere Impres, Capto MMC, Capto MMC Impres, Capto Core 700, PPA Hypercel, HEA Hypercel, MEP Hypercel, Eshmuno HCX, Toyopearl MX-Trp-650M, Nuvia C Prime, CHT Type I, and CHT Type II.

### Partition Coefficient (Kₚ) and Separation Factor (α)

Partition coefficient (Kₚ) and separation factor (α) are two thermodynamic parameters specific for an operating condition of a chromatographic process, which can be used to quantify separation that can be achieved through the process under the operating condition.

A partitioning coefficient, K_{P}, is determined by mixing a known liquid concentration of protein (or other molecule of interest) with a known volume of chromatographic resin and calculating the ratio of the protein bound to the resin and the protein remaining in the liquid at equilibrium: K_{P}=q/c=[bound]/[free].

Partitioning is generally reported in terms of log K_{P}, which can be accurately quantified from approximately 0 to 2 using the UV method described herein. General rules for log K_{P}screening are as follows:
log K_{P}≥1.5, strong binding to the resin;
log K_{P}<1, conditions where elution would be expected for a bind-and-elute modality;
0.5<log K_{P}<1, weak interaction conditions that will show some binding;
log K_{P}<0.5, very little or no binding.

The difference of log Kₚ values between different species can be used to predict separation of the species through the calculation of a separation factor, a, as follows: α=K_{P, protein 1}/K_{P, protein 2}; log α=log K_{P, protein 1}−log K_{P, protein2}, where a log α further from 0 indicates better separation. In certain embodiments, an absolute value of log α larger than 0.2 indicates good separation between the two species. In some embodiments, an absolute value of log α larger than 0.3 indicates good separation between the two species. In other embodiments, an absolute value of log α larger than 0.5 indicates good separation between the two species. In other embodiments, an absolute value of log α larger than 1.0 indicates good separation between the two species.

### HCP

The various methods provided herein apply to a broad variety of HCP. The HCP can be any endogenous protein derived from a host cell (e.g., CHO cell) during bioprocessing of an anti-LAG3 antibody or antigen binding fragment expressed in the host cell. Non-limiting examples of HCP include structural protein, functional protein, secreted protein, enzyme, such as lipase, proteinase, and kinase, etc. In some embodiments, the HCP is a structural protein. In certain embodiments, the HCP is a functional protein. In other embodiments, the HCP is a secreted protein. In yet another embodiment, the HCP is an enzyme. In one embodiment, the HCP is a lipase. In another embodiment, the HCP is a proteinase. In yet another embodiment, the HCP is a kinase. In one embodiment, the HCP is Clusterin.

In certain embodiments, the lipase is selected from the group consisting of PLBL2, LPL, LPLA2, LP-PLA2, and LAL. In one embodiment, the lipase is PLBL2. In another embodiment, the lipase is LPL. In yet another embodiment, the lipase is LPLA2. In one embodiment, the lipase is LP-PLA2. In another embodiment, the lipase is LAL. In still another embodiment, the lipase includes two, three, four, five, six, seven, eight, nine, ten, or more different lipases. In yet still another embodiment, the lipase includes two, three, four, or five different lipases selected from the group consisting of PLBL2, LPL, LPLA2, LP-PLA2, and LAL. In one embodiment, the lipase includes PLBL2 and LPL. In another embodiment, the lipase includes PLBL2 and LPLA2. In yet another embodiment, the lipase includes PLBL2 and LP-PLA2. In still another embodiment, the lipase includes PLBL2 and LAL. In one embodiment, the lipase includes LPL and LPLA2. In another embodiment, the lipase includes LPL and LP-PLA2. In yet another embodiment, the lipase includes LPL and LAL. In still another embodiment, the lipase includes LPLA2 and LP-PLA2. In one embodiment, the lipase includes LPLA2 and LAL. In another embodiment, the lipase includes LP-PLA2 and LAL. In yet another embodiment, the lipase includes PLBL2, LPL, and LPLA2. In still another embodiment, the lipase includes PLBL2, LPL, and LP-PLA2. In one embodiment, the lipase includes PLBL2, LPL, and LAL. In another embodiment, the lipase includes PLBL2, LPLA2, and LP-PLA2. In yet another embodiment, the lipase includes PLBL2, LPLA2, and LAL. In still another embodiment, the lipase includes PLBL2, LP-PLA2, and LAL. In one embodiment, the lipase includes LPL, LPLA2, and LP-PLA2. In another embodiment, the lipase includes LPL, LPLA2, and LAL. In yet another embodiment, the lipase includes LPL, LP-PLA2, and LAL. In still another embodiment, the lipase includes LPLA2, LP-PLA2, and LAL. In one embodiment, the lipase includes PLBL2, LPL, LPLA2, and LP-PLA2. In another embodiment, the lipase includes PLBL2, LPL, LPLA2, and LAL. In yet another embodiment, the lipase includes PLBL2, LPL, LP-PLA2, and LAL. In still another embodiment, the lipase includes PLBL2, LPLA2, LP-PLA2, and LAL. In yet still another embodiment, the lipase includes PLBL2, LPL, LPLA2, LP-PLA2, and LAL.

The host cell can be any cell used for expressing an exogenous protein. Common host cells used in manufacturing of biopharmaceuticals include but are not limited to CHO cell, baby hamster kidney (BHK21) cell, murine myeloma NSO cell, murine myeloma Sp2/0 cell, human embryonic kidney 293 (HEK293) cell, fibrosarcoma HT-1080 cell, PER.C6 cell, HKB-11 cell, CAP cell, HuH-7 cell, murine C127 cell, and a naturally generated or genetically modified variant thereof. In certain embodiments, the host cell is CHO cell. In some embodiments, the host cell is baby hamster kidney (BHK21) cell. In other embodiments, the host cell is murine myeloma NSO cell. In yet other embodiments, the host cell is murine myeloma Sp2/0 cell. In still other embodiments, the host cell is human embryonic kidney 293 (HEK293) cell. In certain embodiments, the host cell is fibrosarcoma HT-1080 cell. In some embodiments, the host cell is PER.C6 cell. In other embodiments, the host cell is HKB-11 cell. In yet other embodiments, the host cell is CAP cell. In still other embodiments, the host cell is HuH-7 cell. In certain embodiments, the host cell is murine C127 cell. In some embodiments, the host cell is a naturally generated variant of the above host cell. In other embodiments, the host cell is a genetically modified variant of the above host cell.

In certain embodiments, the CHO cell lipase is selected from the group consisting of PLBL2, LPL, LPLA2, LP-PLA2, and LAL. In one embodiment, the CHO cell lipase is PLBL2. In another embodiment, the CHO cell lipase is LPL. In yet another embodiment, the CHO cell lipase is LPLA2. In one embodiment, the CHO cell lipase is LP-PLA2. In another embodiment, the CHO cell lipase is LAL. In still another embodiment, the CHO cell lipase includes two, three, four, five, six, seven, eight, nine, ten, or more different CHO cell lipases. In yet still another embodiment, the CHO cell lipase includes two, three, four, or five different CHO cell lipases selected from the group consisting of PLBL2, LPL, LPLA2, LP-PLA2, and LAL. In one embodiment, the CHO cell lipase includes PLBL2 and LPL. In another embodiment, the CHO cell lipase includes PLBL2 and LPLA2. In yet another embodiment, the CHO cell lipase includes PLBL2 and LP-PLA2. In still another embodiment, the CHO cell lipase includes PLBL2 and LAL. In one embodiment, the CHO cell lipase includes LPL and LPLA2. In another embodiment, the CHO cell lipase includes LPL and LP-PLA2. In yet another embodiment, the CHO cell lipase includes LPL and LAL. In still another embodiment, the CHO cell lipase includes LPLA2 and LP-PLA2. In one embodiment, the CHO cell lipase includes LPLA2 and LAL. In another embodiment, the CHO cell lipase includes LP-PLA2 and LAL. In yet another embodiment, the CHO cell lipase includes PLBL2, LPL, and LPLA2. In still another embodiment, the CHO cell lipase includes PLBL2, LPL, and LP-PLA2. In one embodiment, the CHO cell lipase includes PLBL2, LPL, and LAL. In another embodiment, the CHO cell lipase includes PLBL2, LPLA2, and LP-PLA2. In yet another embodiment, the CHO cell lipase includes PLBL2, LPLA2, and LAL. In still another embodiment, the CHO cell lipase includes PLBL2, LP-PLA2, and LAL. In one embodiment, the CHO cell lipase includes LPL, LPLA2, and LP-PLA2. In another embodiment, the CHO cell lipase includes LPL, LPLA2, and LAL. In yet another embodiment, the CHO cell lipase includes LPL, LP-PLA2, and LAL. In still another embodiment, the CHO cell lipase includes LPLA2, LP-PLA2, and LAL. In one embodiment, the CHO cell lipase includes PLBL2, LPL, LPLA2, and LP-PLA2. In another embodiment, the CHO cell lipase includes PLBL2, LPL, LPLA2, and LAL. In yet another embodiment, the CHO cell lipase includes PLBL2, LPL, LP-PLA2, and LAL. In still another embodiment, the CHO cell lipase includes PLBL2, LPLA2, LP-PLA2, and LAL. In yet still another embodiment, the CHO cell lipase includes PLBL2, LPL, LPLA2, LP-PLA2, and LAL.

Methods of Screening Operating Conditions for Separation of a Host Cell Lipase from an Anti-LAG3 Antibody

This disclosure provides methods of screening operating conditions for separation of a HCP (e.g., lipase) from an anti-LAG3 antibody or antigen-binding fragment through the chromatographic process of the invention.

A plethora of operating conditions, including pH, with or without salt, salt type, salt concentration, other components (e.g., counter ion) in solution, concentration of each component, or load protein concentration, etc., can be designed and examined for the HCP (e.g., lipase) or the anti-LAG3 antibody or antigen-binding fragment. Operating conditions to be screened can be commonly used process conditions for the resin selected, for example, equilibration condition, loading condition, washing condition, elution condition, or stripping condition, etc.

The Kₚ values of the HCP (e.g., lipase) and the anti-LAG3 antibody or antigen-binding fragment are determined by methods disclosed herein or commonly understood by a person of ordinary skill in the art. Log α values between the HCP (e.g., lipase) and the anti-LAG3 antibody or antigen-binding fragment are calculated using methods described herein. In general, an absolute value of log α larger than 0.5 is desirable for good separation between the HCP (e.g., lipase) and the anti-LAG3 antibody or antigen-binding fragment.

In one embodiment, the screening is performed using a resin slurry plate method, as disclosed in Welsh et al., BiotechnolProg. 30 (3):626-635 (2014). For example, mixtures of different combinations of pH, salt, and feed are added into 96-well filter plates (e.g., P/N MSBVN1250, Millipore Sigma, Burlington, Mass.). The chromatographic resin volume is 2-50 μL, and the liquid feed volume is 200 μL. In some embodiments, 16-32 conditions are tested for each resin. In other embodiments, 24-96 conditions are tested for each resin. Separation of resin and liquid was accomplished by vacuum filtration. First, the resin is incubated with the equilibration buffer for 10 minutes and the equilibration step is repeated three times. Next, the resin is incubated with feed for 60 minutes. Then, the resin is incubated in strip condition for 10 minutes and repeated twice. The equilibration step allows for buffer exchange from the initial resin slurry buffer. The 60 min time for feed mixing allows for pseudo equilibration between the resin ligand and protein at a given set of conditions. The filtrate from the feed step was measured by UV absorbance at 280-320 nm to determine the final liquid concentration of the protein, c. The bound concentration of the protein, q, was determined by a mass balance of c and the known feed concentration, co.

In another embodiment, the screening is performed using a mini-column method, as disclosed in Welsh et al., Biotechnol Prog. 30 (3):626-635 (2014) or Petroff et al., Biotech Bioeng. 113 (6):1273-1283 (2015). For example, mixtures of different combinations of pH, salt, and feed are screened in a 0.6 mL column format with a 3 cm bed height. Up to 8 columns are screened in parallel. A typical residence time of about 4 min is preserved in the miniature columns by reducing the linear flowrate from about 300 cm/h for a typical column to about 45 cm/h in the miniature column format. All other typical parameters for chromatography screening are conserved. Eluate factions can be collected as pools or as fractions by collecting in 96-well plates to produce chromatograms similar to lab scale studies.

Once the operating conditions for separating the HCP (e.g., lipase) from the anti-LAG3 antibody or antigen binding fragment are determined, the conditions of the load fluid and/or resin can be adjusted accordingly. For example, the resin can be equilibrated by washing it with a solution that will bring it to the necessary operating conditions.

Methods of Separating a Host Cell Lipase from an Anti-LAG3 Antibody

This disclosure further provides methods of separating a HCP (e.g., lipase) from an anti-LAG3 antibody or antigen binding fragments through a chromatographic process.

In one aspect, provided herein is a method of separating a host cell lipase from a composition comprising an anti-LAG3 antibody or antigen-binding fragment and a host cell lipase through a hydrophobic interaction chromatographic (HIC) process, comprising:

(a) passing a load fluid comprising the composition through a HIC resin under a loading operating condition; and

(b) collecting the anti-LAG3 antibody or antigen-binding fragment in a flowthrough; wherein separation factor (α) is the ratio of the partition coefficient (Kₚ) for the lipase to the Kₚ for the anti-LAG3 antibody or antigen-binding fragment, and wherein log α is larger than 0.5 under the loading operating condition; wherein the anti-LAG3 antibody or antigen binding fragment comprises: (a) light chain CDRs of SEQ ID NOs: 6, 7 and 8 and (b) heavy chain CDRs of SEQ ID NOs: 9, 10 and 11.

In another aspect, provided herein is a method of separating a host cell lipase from a composition comprising an anti-LAG3 antibody or antigen-binding fragment and a host cell lipase through a hydrophobic interaction chromatographic (HIC) process, comprising:

(a) passing a load fluid comprising the composition through the HIC resin; and

(b) eluting the anti-LAG3 antibody or antigen-binding fragment from the chromatographic resin with an elution solution under an elution operating condition; wherein separation factor (α) is the ratio of the partition coefficient (Kₚ) for the lipase to the Kₚ for the anti-LAG3 antibody or antigen-binding fragment, and wherein log α is larger than 0.5 under the elution operating condition; wherein the anti-LAG3 antibody or antigen binding fragment comprises: (a) light chain CDRs of SEQ ID NOs: 6, 7 and 8 and (b) heavy chain CDRs of SEQ ID NOs: 9, 10 and 11.

In a further aspect, provided herein is a method of separating a host cell lipase from a composition comprising an anti-LAG3 antibody or antigen-binding fragment and a host cell lipase through a Cation Exchange (CEX) process, comprising:

(a) passing a load fluid comprising the composition through a CEX resin; and

(b) eluting the anti-LAG3 antibody or antigen-binding fragment from the chromatographic resin with an elution solution under an elution operating condition; wherein separation factor (α) is the ratio of the partition coefficient (Kₚ) for the lipase to the Kₚ for the anti-LAG3 antibody or antigen-binding fragment, and wherein log α is larger than 0.5 under the elution operating condition; wherein the anti-LAG3 antibody or antigen binding fragment comprises: (a) light chain CDRs of SEQ ID NOs: 6, 7 and 8 and (b) heavy chain CDRs of SEQ ID NOs: 9, 10 and 11.

In certain embodiments, log α is larger than 1.0 under the loading operating condition.

In some embodiments, the log Kₚ for the lipase is larger than 1.0 under the loading operating condition. In other embodiments, the log Kₚ for the lipase is larger than 1.5 under the loading operating condition.

In certain embodiments, log α is larger than 0.5 and the log Kₚ for the lipase is larger than 1.0 under the loading operating condition. In some embodiments, log α is larger than 0.5 and the log Kₚ for the lipase is larger than 1.5 under the loading operating condition. In other embodiments, log α is larger than 1.0 and the log Kₚ for the lipase is larger than 1.0 under the loading operating condition. In yet other embodiments, log α is larger than 1.0 and the log Kₚ for the lipase is larger than 1.5 under the loading operating condition.

In certain embodiments, the lipase is selected from the group consisting of PLBL2, LPL, LPLA2, LP-PLA2, and LAL. In one embodiment, the lipase is PLBL2. In another embodiment, the lipase is LPL. In yet another embodiment, the lipase is LPLA2. In one embodiment, the lipase is LP-PLA2. In another embodiment, the lipase is LAL. In still another embodiment, the lipase includes two, three, four, five, six, seven, eight, nine, ten, or more different lipases. In yet still another embodiment, the lipase includes two, three, four, or five different lipases selected from the group consisting of PLBL2, LPL, LPLA2, LP-PLA2, and LAL. In one embodiment, the lipase includes PLBL2 and LPL. In another embodiment, the lipase includes PLBL2 and LPLA2. In yet another embodiment, the lipase includes PLBL2 and LP-PLA2. In still another embodiment, the lipase includes PLBL2 and LAL. In one embodiment, the lipase includes LPL and LPLA2. In another embodiment, the lipase includes LPL and LP-PLA2. In yet another embodiment, the lipase includes LPL and LAL. In still another embodiment, the lipase includes LPLA2 and LP-PLA2. In one embodiment, the lipase includes LPLA2 and LAL. In another embodiment, the lipase includes LP-PLA2 and LAL. In yet another embodiment, the lipase includes PLBL2, LPL, and LPLA2. In still another embodiment, the lipase includes PLBL2, LPL, and LP-PLA2. In one embodiment, the lipase includes PLBL2, LPL, and LAL. In another embodiment, the lipase includes PLBL2, LPLA2, and LP-PLA2. In yet another embodiment, the lipase includes PLBL2, LPLA2, and LAL. In still another embodiment, the lipase includes PLBL2, LP-PLA2, and LAL. In one embodiment, the lipase includes LPL, LPLA2, and LP-PLA2. In another embodiment, the lipase includes LPL, LPLA2, and LAL. In yet another embodiment, the lipase includes LPL, LP-PLA2, and LAL. In still another embodiment, the lipase includes LPLA2, LP-PLA2, and LAL. In one embodiment, the lipase includes PLBL2, LPL, LPLA2, and LP-PLA2. In another embodiment, the lipase includes PLBL2, LPL, LPLA2, and LAL. In yet another embodiment, the lipase includes PLBL2, LPL, LP-PLA2, and LAL. In still another embodiment, the lipase includes PLBL2, LPLA2, LP-PLA2, and LAL. In yet still another embodiment, the lipase includes PLBL2, LPL, LPLA2, LP-PLA2, and LAL.

In some embodiments of various methods provided herein, the lipase is a CHO cell lipase. In certain embodiments, the CHO cell lipase is selected from the group consisting of PLBL2, LPL, LPLA2, LP-PLA2, and LAL. In one embodiment, the CHO cell lipase is PLBL2. In another embodiment, the CHO cell lipase is LPL. In yet another embodiment, the CHO cell lipase is LPLA2. In one embodiment, the CHO cell lipase is LP-PLA2. In another embodiment, the CHO cell lipase is LAL. In still another embodiment, the CHO cell lipase includes two, three, four, five, six, seven, eight, nine, ten, or more different CHO cell lipases. In yet still another embodiment, the CHO cell lipase includes two, three, four, or five different CHO cell lipases selected from the group consisting of PLBL2, LPL, LPLA2, LP-PLA2, and LAL. In one embodiment, the CHO cell lipase includes PLBL2 and LPL. In another embodiment, the CHO cell lipase includes PLBL2 and LPLA2. In yet another embodiment, the CHO cell lipase includes PLBL2 and LP-PLA2. In still another embodiment, the CHO cell lipase includes PLBL2 and LAL. In one embodiment, the CHO cell lipase includes LPL and LPLA2. In another embodiment, the CHO cell lipase includes LPL and LP-PLA2. In yet another embodiment, the CHO cell lipase includes LPL and LAL. In still another embodiment, the CHO cell lipase includes LPLA2 and LP-PLA2. In one embodiment, the CHO cell lipase includes LPLA2 and LAL. In another embodiment, the CHO cell lipase includes LP-PLA2 and LAL. In yet another embodiment, the CHO cell lipase includes PLBL2, LPL, and LPLA2. In still another embodiment, the CHO cell lipase includes PLBL2, LPL, and LP-PLA2. In one embodiment, the CHO cell lipase includes PLBL2, LPL, and LAL. In another embodiment, the CHO cell lipase includes PLBL2, LPLA2, and LP-PLA2. In yet another embodiment, the CHO cell lipase includes PLBL2, LPLA2, and LAL. In still another embodiment, the CHO cell lipase includes PLBL2, LP-PLA2, and LAL. In one embodiment, the CHO cell lipase includes LPL, LPLA2, and LP-PLA2. In another embodiment, the CHO cell lipase includes LPL, LPLA2, and LAL. In yet another embodiment, the CHO cell lipase includes LPL, LP-PLA2, and LAL. In still another embodiment, the CHO cell lipase includes LPLA2, LP-PLA2, and LAL. In one embodiment, the CHO cell lipase includes PLBL2, LPL, LPLA2, and LP-PLA2. In another embodiment, the CHO cell lipase includes PLBL2, LPL, LPLA2, and LAL. In yet another embodiment, the CHO cell lipase includes PLBL2, LPL, LP-PLA2, and LAL. In still another embodiment, the CHO cell lipase includes PLBL2, LPLA2, LP-PLA2, and LAL. In yet still another embodiment, the CHO cell lipase includes PLBL2, LPL, LPLA2, LP-PLA2, and LAL.

In certain embodiments of various methods provided herein, the operating condition further comprises modulating ionic strength and/or conductivity by adding a salt. In some embodiments, the effect of adding a salt is to achieve the desired log α. In other embodiments, the effect of adding a salt is to achieve the desired log Kₚ for the lipase. In yet other embodiments, the effect of adding a salt is to achieve the desired log α and the desired log Kₚ for the lipase. Thus, in one embodiment, the operating condition further comprises achieving the desired log α by adding a salt. In another embodiment, the operating condition further comprises achieving the desired log Kₚ for the lipase by adding a salt. In yet another embodiment, the operating condition further comprises achieving the desired log α and the desired log Kₚ for the lipase by adding a salt.

In some embodiments, the salt in the operating solution is selected from the group consisting of sodium chloride, sodium acetate, sodium phosphate, ammonium sulfate, sodium sulfate, and Tris-HCl. In one embodiment, the salt is sodium chloride. In another embodiment, the salt is sodium acetate. In yet another embodiment, the salt is sodium phosphate. In still another embodiment, the salt is ammonium sulfate. In one embodiment, the salt is sodium sulfate. In another embodiment, the salt is Tris-HCl.

In one embodiment, the concentration of sodium chloride in the operating solution is from about 100 mM to about 225 mM, the chromatographic resin is CEX, the pH of the operating condition is from about 4.5 to about 8.0. In another embodiment, the concentration of sodium chloride in the operating solution is from about 150 mM to about 180 mM, the chromatographic resin is CEX, the pH of the operating condition is from about 5.0 to about 8.0. In one embodiment, the concentration of sodium chloride in the operating solution is from about 100 mM to about 225 mM, the chromatographic resin is CEX, the pH of the operating condition is from about 5.0 to about 6.0. In another embodiment, the concentration of sodium chloride in the operating solution is from about 150 mM to about 180 mM, the chromatographic resin is CEX, the pH of the operating condition is from about 5.0 to about 6.0.

In a further aspect, provided herein is a method of separating a PLBL2 or LPLA2 from a composition comprising an anti-LAG3 antibody or antigen-binding fragment and a PLBL2 or LPLA2 through a hydrophobic interaction chromatographic process, comprising:

(a) passing a load fluid comprising the composition through a hydrophobic interaction chromatographic resin; and

(b) collecting the anti-LAG3 antibody or antigen-binding fragment in a flowthrough; and wherein the load fluid has a conductivity of about 25 to 80 mS/cm; wherein the anti-LAG3 antibody or antigen binding fragment comprises: (a) light chain CDRs of SEQ ID NOs: 6, 7 and 8 and (b) heavy chain CDRs of SEQ ID NOs: 9, 10 and 11.

In a further aspect, provided herein is a method of separating a PLBL2 or LPLA2 from a composition comprising an anti-LAG3 antibody or antigen-binding fragment and a PLBL2 or LPLA2 through a hydrophobic interaction chromatographic process, comprising:

(a) passing a load fluid comprising the composition through a HIC resin; and

(b) eluting the anti-LAG3 antibody or antigen-binding fragment from the HIC resin with an elution solution; wherein the elution solution has a conductivity of about 25 to 80 mS/cm; wherein the anti-LAG3 antibody or antigen binding fragment comprises: (a) light chain CDRs of SEQ ID NOs: 6, 7 and 8 and (b) heavy chain CDRs of SEQ ID NOs: 9, 10 and 11.

In still another specific embodiment, the concentration of sodium sulfate in the operating solution is from about 500 mM to about 620 mM, the chromatographic resin is HIC, and the pH of the operating condition is about 7. In yet still another specific embodiment, the concentration of sodium sulfate in the operating solution is from about 510 mM to about 560 mM, the chromatographic resin is HIC, and the pH of the operating condition is about 7.

In one aspect of the HIC chromatographic process, the load fluid or elution solution has a conductivity of about 50 to 70 mS/cm. In another embodiment, the load fluid or elution solution comprises about 300 mM to about 650 mM monovalent or divalent salt. In another embodiment, the load fluid or elution solution comprises about 300 mM to about 650 mM monovalent or divalent salt, and the pH is 4.5-7.5. In another embodiment, the salt is about 500-620 mM sodium sulfate, and the pH is about 5-7.5. In a further embodiment, the salt is 560 mM sodium sulfate, and the pH of the load fluid or elution solution is about 7.

The methods of separation provided herein can be used in combination with one or more separation steps described herein or commonly used in the art. In one embodiment, one or more separation steps precede the method described herein. In another embodiment, one or more separation steps follow the method described herein. In yet another embodiment, one or more separation steps are performed between two methods described herein. In still other embodiments, one or more separation steps are performed before, after, and/or between the methods described herein. There is no limitation of how many separation steps or methods can be combined or the order of the separation steps or methods to be combined.

In more embodiments of the various methods provided herein, the load fluid is an eluate from a prior chromatographic process. In one embodiment, the prior chromatographic process comprises an affinity chromatography. In another embodiment, the prior chromatographic process comprises an affinity chromatography followed by an ion exchange chromatography. In yet another embodiment, the affinity chromatography is a protein A chromatography. In still another embodiment, the ion exchange chromatography is an AEX chromatography. In yet still another embodiment, the prior chromatographic process comprises a protein A chromatography followed by an AEX chromatography.

### Methods of Improving PS-80 Stability in an Anti-LAG3 Antibody Formulation

This disclosure further provides methods of improving PS-80 stability in an anti-LAG3 antibody or antigen binding fragment formulation (e.g., drug substance formulation or drug product formulation) by separating a HCP (e.g., lipase) from the anti-LAG3 antibody or antigen binding fragment using a chromatographic process.

In yet still another aspect, provided herein is a method of improving polysorbate-80 (PS-80) stability in an anti-LAG3 antibody or antigen-binding fragment formulation, comprising:

(a) passing a load fluid comprising a host cell lipase and the anti-LAG3 antibody or antigen-binding fragment through a HIC resin under a loading operating condition;

(b) collecting the anti-LAG3 antibody or antigen-binding fragment in a flowthrough; and

(c) formulating the anti-LAG3 antibody or antigen-binding fragment so that the anti-LAG3 antibody or antigen-binding fragment formulation is a PS-80-containing solution;

wherein separation factor (α) is the ratio of the partition coefficient (Kₚ) for the lipase to the Kₚ for the anti-LAG3 antibody or antigen-binding fragment, and wherein log α is larger than 0.5 under the loading operating condition; wherein the anti-LAG3 antibody or antigen binding fragment comprises: (a) light chain CDRs of SEQ ID NOs: 6, 7 and 8 and (b) heavy chain CDRs of SEQ ID NOs: 9, 10 and 11. The improvement in PS-80 stability is for steps (a), (b) and (c) as compared to step (c) alone.

In yet still another aspect, provided herein is a method of formulating an anti-LAG3 antibody or antigen-binding fragment formulation, comprising:
(a) passing a load fluid comprising a host cell lipase and the anti-LAG3 antibody or antigen-binding fragment through a HIC resin under a loading operating condition;
(b) collecting the anti-LAG3 antibody or antigen-binding fragment in a flowthrough; and
(c) formulating the anti-LAG3 antibody or antigen-binding fragment by adding PS-80 to the formulation;
   wherein separation factor (α) is the ratio of the partition coefficient (Kₚ) for the lipase to the Kₚ for the anti-LAG3 antibody or antigen-binding fragment, and wherein log α is larger than 0.5 under the loading operating condition; wherein the anti-LAG3 antibody or antigen binding fragment comprises: (a) light chain CDRs of SEQ ID NOs: 6, 7 and 8 and (b) heavy chain CDRs of SEQ ID NOs: 9, 10 and 11.

In certain embodiments, log α is larger than 1.0 under the loading operating condition.

In some embodiments, the log Kₚ for the lipase is larger than 1.0 under the loading operating condition. In other embodiments, the log Kₚ for the lipase is larger than 1.5 under the loading operating condition.

In certain embodiments, log α is larger than 0.5 and the log Kₚ for the lipase is larger than 1.0 under the loading operating condition. In some embodiments, log α is larger than 0.5 and the log Kₚ for the lipase is larger than 1.5 under the loading operating condition. In other embodiments, log α is larger than 1.0 and the log Kₚ for the lipase is larger than 1.0 under the loading operating condition. In yet other embodiments, log α is larger than 1.0 and the log Kₚ for the lipase is larger than 1.5 under the loading operating condition.

In another aspect, provided herein is a method of improving PS-80 stability in an anti-LAG3 antibody formulation, comprising:

(a) passing a load fluid comprising a host cell lipase and the anti-LAG3 antibody through a HIC resin;

(b) eluting the anti-LAG3 antibody from the chromatographic resin with an elution solution under an elution operating condition; and

(c) formulating the anti-LAG3 antibody so that the anti-LAG3 antibody formulation is a PS-80-containing solution;

wherein α is the ratio of Kₚ for the lipase to the Kₚ for the anti-LAG3 antibody, and wherein log α is larger than 0.5 under the elution operating condition. The improvement in PS-80 stability is for steps (a), (b) and (c) as compared to step (c) alone.

In another aspect, provided herein is a method of formulating an anti-LAG3 antibody formulation, comprising:
(a) passing a load fluid comprising a host cell lipase and the anti-LAG3 antibody through a HIC resin;
(b) eluting the anti-LAG3 antibody from the chromatographic resin with an elution solution under an elution operating condition; and
(c) formulating the anti-LAG3 antibody by adding PS-80 in the formulation;
   wherein α is the ratio of Kₚ for the lipase to the Kₚ for the anti-LAG3 antibody, and wherein log α is larger than 0.5 under the elution operating condition.

In certain embodiments, log α is larger than 1.0 under the elution operating condition.

In some embodiments, the log Kₚ for the lipase is larger than 1.0 under the elution operating condition. In other embodiments, the log Kₚ for the lipase is larger than 1.5 under the elution operating condition.

In certain embodiments, log α is larger than 0.5 and the log Kₚ for the lipase is larger than 1.0 under the elution operating condition. In some embodiments, log α is larger than 0.5 and the log Kₚ for the lipase is larger than 1.5 under the elution operating condition. In other embodiments, log α is larger than 1.0 and the log Kₚ for the lipase is larger than 1.0 under the elution operating condition. In yet other embodiments, log α is larger than 1.0 and the log Kₚ for the lipase is larger than 1.5 under the elution operating condition.

In certain embodiments, the lipase is selected from the group consisting of PLBL2, LPL, LPLA2, LP-PLA2, and LAL. In one embodiment, the lipase is PLBL2. In another embodiment, the lipase is LPL. In yet another embodiment, the lipase is LPLA2. In one embodiment, the lipase is LP-PLA2. In another embodiment, the lipase is LAL. In still another embodiment, the lipase includes two, three, four, five, six, seven, eight, nine, ten, or more different lipases. In yet still another embodiment, the lipase includes two, three, four, or five different lipases selected from the group consisting of PLBL2, LPL, LPLA2, LP-PLA2, and LAL. In one embodiment, the lipase includes PLBL2 and LPL. In another embodiment, the lipase includes PLBL2 and LPLA2. In yet another embodiment, the lipase includes PLBL2 and LP-PLA2. In still another embodiment, the lipase includes PLBL2 and LAL. In one embodiment, the lipase includes LPL and LPLA2. In another embodiment, the lipase includes LPL and LP-PLA2. In yet another embodiment, the lipase includes LPL and LAL. In still another embodiment, the lipase includes LPLA2 and LP-PLA2. In one embodiment, the lipase includes LPLA2 and LAL. In another embodiment, the lipase includes LP-PLA2 and LAL. In yet another embodiment, the lipase includes PLBL2, LPL, and LPLA2. In still another embodiment, the lipase includes PLBL2, LPL, and LP-PLA2. In one embodiment, the lipase includes PLBL2, LPL, and LAL. In another embodiment, the lipase includes PLBL2, LPLA2, and LP-PLA2. In yet another embodiment, the lipase includes PLBL2, LPLA2, and LAL. In still another embodiment, the lipase includes PLBL2, LP-PLA2, and LAL. In one embodiment, the lipase includes LPL, LPLA2, and LP-PLA2. In another embodiment, the lipase includes LPL, LPLA2, and LAL. In yet another embodiment, the lipase includes LPL, LP-PLA2, and LAL. In still another embodiment, the lipase includes LPLA2, LP-PLA2, and LAL. In one embodiment, the lipase includes PLBL2, LPL, LPLA2, and LP-PLA2. In another embodiment, the lipase includes PLBL2, LPL, LPLA2, and LAL. In yet another embodiment, the lipase includes PLBL2, LPL, LP-PLA2, and LAL. In still another embodiment, the lipase includes PLBL2, LPLA2, LP-PLA2, and LAL. In yet still another embodiment, the lipase includes PLBL2, LPL, LPLA2, LP-PLA2, and LAL.

In some embodiments of various methods provided herein, the lipase is a Chinese Hamster Ovary (CHO) cell lipase. In certain embodiments, the CHO cell lipase is selected from the group consisting of PLBL2, LPL, LPLA2, LP-PLA2, and LAL. In one embodiment, the CHO cell lipase is PLBL2. In another embodiment, the CHO cell lipase is LPL. In yet another embodiment, the CHO cell lipase is LPLA2. In one embodiment, the CHO cell lipase is LP-PLA2. In another embodiment, the CHO cell lipase is LAL. In still another embodiment, the CHO cell lipase includes two, three, four, five, six, seven, eight, nine, ten, or more different CHO cell lipases. In yet still another embodiment, the CHO cell lipase includes two, three, four, or five different CHO cell lipases selected from the group consisting of PLBL2, LPL, LPLA2, LP-PLA2, and LAL. In one embodiment, the CHO cell lipase includes PLBL2 and LPL. In another embodiment, the CHO cell lipase includes PLBL2 and LPLA2. In yet another embodiment, the CHO cell lipase includes PLBL2 and LP-PLA2. In still another embodiment, the CHO cell lipase includes PLBL2 and LAL. In one embodiment, the CHO cell lipase includes LPL and LPLA2. In another embodiment, the CHO cell lipase includes LPL and LP-PLA2. In yet another embodiment, the CHO cell lipase includes LPL and LAL. In still another embodiment, the CHO cell lipase includes LPLA2 and LP-PLA2. In one embodiment, the CHO cell lipase includes LPLA2 and LAL. In another embodiment, the CHO cell lipase includes LP-PLA2 and LAL. In yet another embodiment, the CHO cell lipase includes PLBL2, LPL, and LPLA2. In still another embodiment, the CHO cell lipase includes PLBL2, LPL, and LP-PLA2. In one embodiment, the CHO cell lipase includes PLBL2, LPL, and LAL. In another embodiment, the CHO cell lipase includes PLBL2, LPLA2, and LP-PLA2. In yet another embodiment, the CHO cell lipase includes PLBL2, LPLA2, and LAL. In still another embodiment, the CHO cell lipase includes PLBL2, LP-PLA2, and LAL. In one embodiment, the CHO cell lipase includes LPL, LPLA2, and LP-PLA2. In another embodiment, the CHO cell lipase includes LPL, LPLA2, and LAL. In yet another embodiment, the CHO cell lipase includes LPL, LP-PLA2, and LAL. In still another embodiment, the CHO cell lipase includes LPLA2, LP-PLA2, and LAL. In one embodiment, the CHO cell lipase includes PLBL2, LPL, LPLA2, and LP-PLA2. In another embodiment, the CHO cell lipase includes PLBL2, LPL, LPLA2, and LAL. In yet another embodiment, the CHO cell lipase includes PLBL2, LPL, LP-PLA2, and LAL. In still another embodiment, the CHO cell lipase includes PLBL2, LPLA2, LP-PLA2, and LAL. In yet still another embodiment, the CHO cell lipase includes PLBL2, LPL, LPLA2, LP-PLA2, and LAL.

In certain embodiments of various methods provided herein, the operating condition further comprises modulating the ionic strength and/or conductivity of the operating solution by adding a salt. In one embodiment, the operating condition further comprises modulating the ionic strength of the operating solution by adding a salt. In another embodiment, the operating condition further comprises modulating the conductivity of the operating solution by adding a salt. In yet another embodiment, the operating condition further comprises modulating the ionic strength and conductivity of the operating solution by adding a salt. In some embodiments, the effect of adding a salt is to achieve the desired log α. In other embodiments, the effect of adding a salt is to achieve the desired log Kₚ for the lipase. In yet other embodiments, the effect of adding a salt is to achieve the desired log α and the desired log Kₚ for the lipase.

In some embodiments, the salt in the operating solution is selected from the group consisting of sodium chloride, sodium acetate, sodium phosphate, ammonium sulfate, sodium sulfate, and Tris-HCl. In one embodiment, the salt is sodium chloride. In another embodiment, the salt is sodium acetate. In yet another embodiment, the salt is sodium phosphate. In still another embodiment, the salt is ammonium sulfate. In one embodiment, the salt is sodium sulfate. In another embodiment, the salt is Tris-HCl.

In still another specific embodiment, the concentration of sodium sulfate in the operating solution is from about 500 mM to about 620 mM, the chromatographic resin is HIC, and the pH of the operating condition is about 7.

In yet still another specific embodiment, the concentration of sodium sulfate in the operating solution is from about 510 mM to about 560 mM, the chromatographic resin is HIC, and the pH of the operating condition is about 7.

In more embodiments of the various methods provided herein, the load fluid is an eluate from a prior chromatographic process. In one embodiment, the prior chromatographic process comprises an affinity chromatography. In another embodiment, the prior chromatographic process comprises an affinity chromatography followed by a non-affinity chromatography. In yet another embodiment, the affinity chromatography is a protein A chromatography. In still another embodiment, the non-affinity chromatography is an AEX chromatography. In yet still another embodiment, the prior chromatographic process comprises a protein A chromatography followed by an AEX chromatography. In one embodiment, the load fluid is an eluate from a protein A chromatography performed in bind and elute mode followed by AEX chromatography performed in flowthrough mode.

### Pharmaceutical Compositions

The disclosure also provides pharmaceutical compositions comprising an anti-LAG3 antibody or antigen-binding fragment and less than 2 ppm of a host cell lipase, wherein the anti-LAG3 antibody or antigen binding fragment comprises: (a) light chain CDRs of SEQ ID NOs: 6, 7 and 8 and (b) heavy chain CDRs of SEQ ID NOs: 9, 10 and 11.

In certain embodiments, the pharmaceutical composition comprises the anti-LAG3 antibody or antigen-binding fragment and less than 1 ppm of a host cell lipase. In other embodiments, the pharmaceutical composition comprises the anti-LAG3 antibody or antigen-binding fragment and less than 0.1, 0.2, 0.3, 0.4, 0.5, 0.6, 0.7, 0.8, or 0.9 ppm of a host cell lipase. In one embodiment, the pharmaceutical composition comprises the anti-LAG3 antibody or antigen-binding fragment and less than 0.1 ppm of a host cell lipase. In another embodiment, the pharmaceutical composition comprises the anti-LAG3 antibody or antigen-binding fragment and less than 0.2 ppm of a host cell lipase. In yet another embodiment, the pharmaceutical composition comprises the anti-LAG3 antibody or antigen-binding fragment and less than 0.3 ppm of a host cell lipase. In still another embodiment, the pharmaceutical composition comprises the anti-LAG3 antibody or antigen-binding fragment and less than 0.4 ppm of a host cell lipase. In yet still another embodiment, the pharmaceutical composition comprises the anti-LAG3 antibody or antigen-binding fragment and less than 0.5 ppm of a host cell lipase. In one embodiment, the pharmaceutical composition comprises the anti-LAG3 antibody or antigen-binding fragment and less than 0.6 ppm of a host cell lipase. In another embodiment, the pharmaceutical composition comprises the anti-LAG3 antibody or antigen-binding fragment and less than 0.7 ppm of a host cell lipase. In yet another embodiment, the pharmaceutical composition comprises the anti-LAG3 antibody or antigen-binding fragment and less than 0.8 ppm of a host cell lipase. In still another embodiment, the pharmaceutical composition comprises the anti-LAG3 antibody or antigen-binding fragment and less than 0.9 ppm of a host cell lipase.

In certain embodiments of the pharmaceutical compositions, the lipase is selected from the group consisting of PLBL2, LPL, LPLA2, LP-PLA2, and LAL. In one embodiment, the lipase is PLBL2. In another embodiment, the lipase is LPL. In yet another embodiment, the lipase is LPLA2. In one embodiment, the lipase is LP-PLA2. In another embodiment, the lipase is LAL. In still another embodiment, the lipase includes two, three, four, five, six, seven, eight, nine, ten, or more different lipases. In yet still another embodiment, the lipase includes two, three, four, or five different lipases selected from the group consisting of PLBL2, LPL, LPLA2, LP-PLA2, and LAL. In one embodiment, the lipase includes PLBL2 and LPL. In another embodiment, the lipase includes PLBL2 and LPLA2. In yet another embodiment, the lipase includes PLBL2 and LP-PLA2. In still another embodiment, the lipase includes PLBL2 and LAL. In one embodiment, the lipase includes LPL and LPLA2. In another embodiment, the lipase includes LPL and LP-PLA2. In yet another embodiment, the lipase includes LPL and LAL. In still another embodiment, the lipase includes LPLA2 and LP-PLA2. In one embodiment, the lipase includes LPLA2 and LAL. In another embodiment, the lipase includes LP-PLA2 and LAL. In yet another embodiment, the lipase includes PLBL2, LPL, and LPLA2. In still another embodiment, the lipase includes PLBL2, LPL, and LP-PLA2. In one embodiment, the lipase includes PLBL2, LPL, and LAL. In another embodiment, the lipase includes PLBL2, LPLA2, and LP-PLA2. In yet another embodiment, the lipase includes PLBL2, LPLA2, and LAL. In still another embodiment, the lipase includes PLBL2, LP-PLA2, and LAL. In one embodiment, the lipase includes LPL, LPLA2, and LP-PLA2. In another embodiment, the lipase includes LPL, LPLA2, and LAL. In yet another embodiment, the lipase includes LPL, LP-PLA2, and LAL. In still another embodiment, the lipase includes LPLA2, LP-PLA2, and LAL. In one embodiment, the lipase includes PLBL2, LPL, LPLA2, and LP-PLA2. In another embodiment, the lipase includes PLBL2, LPL, LPLA2, and LAL. In yet another embodiment, the lipase includes PLBL2, LPL, LP-PLA2, and LAL. In still another embodiment, the lipase includes PLBL2, LPLA2, LP-PLA2, and LAL. In yet still another embodiment, the lipase includes PLBL2, LPL, LPLA2, LP-PLA2, and LAL.

The disclosure also provides a pharmaceutical composition comprising an anti-LAG3 antibody or antigen-binding fragment and polysorbate 80 (PS80) or polysorbate 20 (PS20), wherein at 1, 3, 6, 9 or 12 months at 2-8° C., the concentration of PS80 or PS20 is maintained at ≥90%, 95% or 99% of the concentration when formulated, wherein the anti-LAG3 antibody or antigen binding fragment comprises: (a) light chain CDRs of SEQ ID NOs: 6, 7 and 8 and (b) heavy chain CDRs of SEQ ID NOs: 9, 10 and 11. In one embodiment, the pharmaceutical composition comprises the anti-LAG3 antibody or antigen-binding fragment and about 0.2 mg/ml of polysorbate 80 (PS80) or polysorbate 20 (PS20) when formulated, wherein at 1, 3, 6, 9 or 12 months at 2-8° C., the concentration of PS80 or PS20 is maintained at least at about 0.18 mg/ml. In one embodiment, PS80 is used in the formulation. In one embodiment, at 1, 3, 6, 9 or 12 months at 2-8° C., PS80 is maintained at ≥95% of the concentration when formulated. In one embodiment, PS80 is maintained at ≥99% of the concentration when formulated.

The disclosure also provides a pharmaceutical composition that comprises about 20.0 mg/mL of the anti-LAG3 antibody or antigen-binding fragment, about 5.0 mg/mL pembrolizumab, about 54 mg/mL sucrose; about 0.2 mg/mL polysorbate 80, about 10 mM histidine buffer at pH about 5.8; about 56 mM L-arginine; and about 8 mM L-methionine when formulated; or a pharmaceutical composition that comprises about 25.0 mg/mL of the anti-LAG3 antibody or antigen-binding fragment; about 50 mg/mL sucrose; about 0.2 mg/mL polysorbate 80; about 10 mM histidine buffer at pH about 5.8; about 70 mM L-Arginine-HCl; and optionally about 10 mM L-methionine when formulated, wherein at 1, 3, 6, 9 or 12 months at 2-8° C., the concentration of PS80 is maintained at least 90%, 95%, 99%, 85%, or 80% of the concentration when formulated.

In various embodiments of the pharmaceutical compositions described herein, the level of the host cell lipase is measured by liquid chromatography-mass spectrometry (LC-MS) or liquid chromatography-Multiple Reaction Monitoring(LC-MRM-MS).

In some embodiments, the pharmaceutical composition is obtainable by a HIC chromatography process comprising the step of:

(a) passing a load fluid comprising the composition through a HIC resin; and

(b) eluting the anti-LAG3 antibody or antigen-binding fragment thereof with an elution solution with a pH from about 5 to about 7.5, and a conductivity of about 25-80 mS/cm; or

(c) collecting the anti-LAG3 antibody or antigen-binding fragment thereof in the flowthrough using loading operation conditions with a pH from about 5 to about 7.5, and a conductivity of about 25-80 mS/cm.

In other embodiments, the pharmaceutical composition is obtainable by a HIC chromatography process comprising the step of:

(a) passing a load fluid comprising the composition through a HIC resin; and

(b) eluting the anti-LAG3 antibody or antigen-binding fragment thereof with an elution solution with a pH from about 5 to about 7.5, and a conductivity of about 50-70 mS/cm; or

(c) collecting the anti-LAG3 antibody or antigen-binding fragment thereof in the flowthrough using loading operation conditions with a pH from about 5 to about 7.5, and a conductivity of about 50-70 mS/cm.

In other embodiments, the HIC chromatography is preceded by Protein A chromatography operated in bind and elute mode and an AEX chromatography operated in a flowthrough mode.

### EXAMPLES

The examples in this section (section VI) are offered by way of illustration, and not by way of limitation.

### Example 1: Method for Determining K_{P} of Different Species

A partitioning coefficient, K_{P}, is determined by mixing a known liquid concentration of protein (or other molecule of interest) with a known volume of chromatography resin and calculating the ratio of the protein bound to the resin and the protein remaining in the liquid: K_{P}=q/c=[bound]/[free].

For subsequent examples 2-3, the chromatography volume was 20 μL, and the liquid volume was 200 μL with a protein concentration of 0.5 mg/mL. These volumes provide a phase ratio of 10:1 for an effective resin loading of 5 mg/mL.

Screenings were conducted by vigorous mixing of resin and liquid in a 96-well filter plate (P/N MSBVN1250, Millipore Sigma, Burlington, Mass.) with separation of resin and liquid by vacuum filtration. The sequence of steps was as follows:

(a) 3× equilibration (buffer not containing feed), 10 min incubation each step;

(b) 1× feed mixing, 60 min incubation; and

(c) 2× strip conditions, 10 min incubation each step

The equilibration step allows for buffer exchange from the initial resin slurry buffer. The 60 min time for feed mixing allows for pseudo equilibration between the resin ligand and protein at a given set of conditions. The filtrate from the feed step was measured by UV absorbance at 280-320 nm to determine the final liquid concentration of the protein, c. The bound concentration of the protein, q, was determined by a mass balance around c and the known feed concentration, c₀ (0.5 mg/mL).

Partitioning is generally reported in terms of log K_{P}, which can be accurately quantified from approximately 0 to 2 using the UV method described here. General rules for log K_{P}screening are as follows:

log K_{P}≥1.5, strong binding to the resin;

log K_{P}<1, conditions where elution would be expected for a bind-and-elute modality;

0.5<log K_{P}<1, weak interaction conditions that will show some binding;

log K_{P}<0.5, very little or no binding.

The log Kₚ of different species is also used to predict separation of different species through the calculation of a separation factor, α, as follows: α=K_{P, protein1}/K_{P, protein2}; log α=log K_{P, protein1}−log K_{P, protein2}, where a log α further from 0 indicates better separation. In the following examples, α=K_{P, lipase}/K_{P, mAb}; log α=log K_{P, lipase}−log K_{P, mAb}. A log α larger than 0.5 indicates good separation between the lipase and a monoclonal antibody. A log α less than −0.5 also indicates good separation between the lipase and a monoclonal antibody.

### Example 2: Comparison of PLBL2 and mAb K_{P} values at typical processing conditions

The method for determining K_{P} and a was used to assess the capability of separating a known lipase impurity, PLBL2, at operating conditions for anti-LAG3 antibody Ab6, through a variety of chromatographic processes.

Table 2 summarizes the log Kp and log α values for Ab6 and PLBL2 at several process conditions for Ab6.

TABLE 2
log K_{P} and log α values for Ab6 and PLBL2
at processing conditions for Ab6

| Process, Resin | Operating Condition | log α |
|---|---|---|
| Protein A, | Equil/wash: 10 mM NaPhosphate, pH 6.5 | −2 |
| MabSelect Sure | High salt wash: 10 mM NaPhosphate, pH | −2 |
| | 6.5, 0.5M NaCl | |
| | Elute: 20 mM NaAcetate, pH 3.5 | −0.3 |
| | Strip: 100 mM acetic acid | 0 |
| AEX, Poros | Load: 100 mM NaAcetate, 100 mM Tris, | −0.5 |
| HQ 50 | pH 7.0 | |
| | Wash: 25 mM NaPhosphate, pH 7.0, 5 | −0.8 |
| | mM NaCl | |
| | Strip: 1M NaCl | 0 |
| CEX, Poros | Load: 100 mM NaAcetate, 100 mM Tris, | 0 |
| HS 50 | pH 5.1 | |
| | Elute (low salt limit): 20 mM NaAcetate, | 1.6 |
| | pH 5.1, 125 mM NaCl | |
| | Elute (center point): 20 mM NaAcetate, | 1.6 |
| | pH 5.1, 150 mM NaCl | |
| | Elute (high salt limit): 20 mM | 1.4 |
| | NaAcetate, pH 5.1, 175 mM NaCl | |
| | Strip: 1M NaCl | 0 |
| HIC, Tosoh- | Load: 25 mM NaPhosphate, 1.13M | 0 |
| Butyl-650M | NaSulfate | |
| (Tosoh Corp., | Elute (low salt limit): 25 mM | 0.3 |
| Tokyo, Japan) | NaPhosphate, pH 7.0, 610 mM NaSulfate | |
| | Elute (low salt limit): 25 mM | 0.9 |
| | NaPhosphate, pH 7.0, 560 mM NaSulfate | |
| | Elute (low salt limit): 25 mM | 1.3 |
| | NaPhosphate, pH 7.0, 510 mM NaSulfate | |
| | Strip: Water | 0 |

For the protein A process, PLBL2 has no affinity, so the majority of PLBL2 would be expected to flow through the protein A resin during loading or wash steps. The only PLBL2 present in pools would likely be from insufficient washes or associated with Ab6.

For the CEX process, Ab6 has lower binding at lower salt and therefore a more robust log α throughout the salt range. For the AEX process, Ab6 binds stronger to the resin than PLBL2, resulting in a negative log α at load and wash conditions. This indicates no separation potential if operating in flowthrough mode and could even indicate enrichment of PLBL2 in the flowthrough due to the stronger binding of Ab6.

An additional HIC process was also tested for Ab6. For this process, PLBL2 had a higher log K_{P} throughout the load and elution conditions and larger log α values at the lower range of the elution salt concentration. This indicates that both Ab6 recovery and PLBL2 separation will be more favorable at these lower salt conditions.

### Example 3: Mapping of PLBL2 and LPLA2 K_{P} Values at a Range of Conditions for HIC Resin

The partitioning coefficient of PLBL2 and LPLA2 for HIC resins with different buffers and conditions that might potentially be used in downstream processing of Ab6 (mAb2) and mAb3 was performed (Table 3).

TABLE 3
Conditions screened for mapping PLBL2 or LPLA2 log Kₚ

| Modality | Buffer | Salt | Salt Concentration (mM) | pH |
|---|---|---|---|---|
| HIC | Phosphate | sodium | 0, 100, 150, 200, 250, 300, | 7.0 |
| | | sulfate | 350, 400, 450, 500, 550, 600 | |

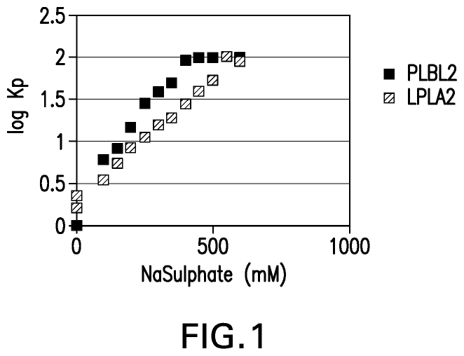

Testing for partitioning of PLBL2 and LPLA2 to a HIC resin, Tosoh Butyl-650M, was conducted by modulating sodium sulfate concentration at a buffering condition of 20 mM sodium phosphate (pH 7.0) (Table 3, FIG. 1). Both lipases showed typical HIC behavior with strong binding at high salt (log K_{P}>1.5 above 250 mM sodium sulfate for PLBL2 and 400 mM sodium sulfate for LPLA2) and decreased partitioning at lower salt (log K_{P}<1 below 150 mM sodium sulfate for PLBL2 and 200 mM sodium sulfate for LPLA2).

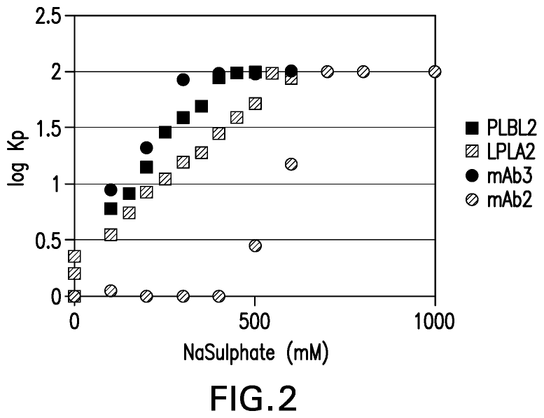

Partitioning of antibodies and lipases was also compared on a HIC resin, Tosoh Butyl-650M, (FIG. 2) at the conditions listed in Table 3. Varying sodium sulfate concentration provides little separation between the mAb3 and PLBL2 with only 300 mM sodium sulfate providing any separation at all with a log α of approximately 0.3 at this condition. LPLA2 provides somewhat better separation with log α of about 0.5 between 300-400 mM sodium sulfate. In contrast, Ab6 is much less hydrophobic than mAb3, PLBL2, or LPLA2, and thus does not transition to strong binding to the HIC resin above log K_{P} of 1.5 until greater than 600 mM sodium sulfate. For Ab6 and PLBL2, log α values from 1.5-2.0 can be achieved between 300-500 mM sodium sulfate, a very wide salt range with promising separation capabilities for operating within. Similarly for LPLA2, log α values greater than 1 are seen in this same salt range.

### Example 4: Hydrophobic Interaction Chromatography Purification of Anti-LAG3 Antibody Preparation with Flowthrough Method

Harvest cell culture fluid containing Ab6 underwent Protein A Affinity chromatography and Anion Ion Exchange chromatography as described in Example 2, and hydrophobic interaction chromatography. The hydrophobic interaction chromatography (Tosoh Toyopearl Butyl-650M) step was operated in flowthrough mode at room temperature, with a target loading of 150 g/L resin. The Viral Filtration Product containing anti-LAG3 antibody Ab6 was adjusted to 560 mM Na₂SO₄ with 1.4 M Na₂SO₄, 1 kg Viral Filtered Product to 0.77 kg 1.4 M Na₂SO₄. Post 1.4 M Na₂SO₄ addition the feed is titrated to a target pH of 7.0 with 1 M Tris base, resulting in the HIC load. Table 4 details the operating steps and parameters for the HIC chromatography: column equilibration, HIC chromatography process. The column effluent absorbance was monitored on-line at a wavelength of 280 nm and used to collect the unadjusted HIC product. The unadjusted HIC product was titrated to a target pH of 5.8 with 1 M Acetic Acid solution. Post pH adjustment, 1 kg of HIC Product was diluted with 2 kg 10 mM Histidine, 70 mM Arginine pH 5.8 and filtered through a Millipore SHC 0.5/0.2 μm filter resulting in the Ultrafiltrated Difiltrated (UFDF) load.

TABLE 4
Summary of the HIC Processing Steps

| | | | Buffer | |
|---|---|---|---|---|
| | | | Conductivity | Buffer |
| Step | Buffer | Flow | (mS/cm) | pH |
| Equilibration | 25 mM NaPhosphate, | Down | 62 ± 5.0 | 7.0 ± |
| | 560 mM Na₂SO₄ | | | 0.2 |
| Load | HIC Column Load | Down | 62 ± 5.0 | 7.0 ± |
| | | | | 0.2 |
| Wash | 25 mM Na Phosphate, | Down | 62 ± 5.0 | 7.0 ± |
| | 560 mM Na₂SO₄ | | | 0.2 |

The in-process intermediates of the above batch along with the corresponding chromatography strip samples were tested for lipase identification by liquid chromatography-multiple reaction monitoring (LC-MRM-MS) as described below (Table 5). PLBL2 was found in the load samples but absent from the HIC flowthrough samples.

A reversed-phase ultra-performance liquid chromatography coupled with multiple reaction monitoring mass spectrometry (RP-UPLC-MRM MS) method was developed on Waters TQS triple quadrupole MS to quantify CHO lipases PLBL2 and LPLA2. The 8-min LC-MRM MS method is a lipase-specific quantitation assay that provides absolute quantitation of the two lipases in bioprocess intermediates and/or in biologics drug substances (ng/mg or ppm). The assay quantitation range of 1-500 ng/mg of each lipase is achieved by spiking CHO recombinant PLBL2 and LPLA2 (MyBioSource) into Ab6 drug substance as protein standards and C13- and N15-heavy labeled peptides of PLBL2 (H₂N-LTFPTGR(¹³C6, ¹⁵N4-OH)) SEQ ID NO: 12 and LPLA2 (H₂N-IPVIGPLK(¹³C6, ¹⁵N2)-OH SEQ ID NO: 13) (New England Peptide) as internal standards (IS). Samples and protein standards were denatured, S—S bond reduced and alkylated, and digested by trypsin before LC-MS analysis. The digested samples were loaded on to a Waters Acquity UPLC BEH C18 column (50×2.1 mm, 1.7 m) and separated by a gradient of 10 to 35% mobile phase B (0.1% formic acid in acetonitrile) at a flow rate of 0.2 mL/min. Mobile phase A was 0.1% formic acid in water. MRM transitions of surrogate peptides generated by trypsin digestion, m/z 396.5 (precursor ion)->m/z 430.3 (fragment ion) of PLBL2 peptide LTFPTGR (SEQ ID NO:12) and m/z 419.1 (precursor ion)->m/z362.3 (fragment ion) of LPLA2 peptide IPVIGPLK (SEQ ID NO:13), were used for PLBL2 and LPLA2 quantitation through respective calibration curves (peak area ratio (Analyte/IS) vs. Analyte concentration) and a weighing factor of 1/x² for linear regression).

TABLE 5
Relative quantification of endogenous PLBL2
in Ab6 process intermediates

| Sample | PLBL2 (ng/mg) |
|---|---|
| Viral Filtration Product before HIC Load | 183.2 |
| HIC Pool (510, 560, 610 | Not detectable |
| mM sodium sulfate) | |

### Example 5: Hydrophobic Interaction Chromatography Purification of Anti-LAG3 Antibody Preparation with Bind and Elute Method

Harvest cell culture fluid containing Ab6 underwent Protein A Affinity chromatography and Anion Ion Exchange chromatography as described in Example 2, and hydrophobic interaction chromatography. The hydrophobic interaction chromatography step (Toyopearl Butyl-650M resin from Tosoh™) was operated in bind and elute mode at room temperature, with a target loading of 30 g/L resin. The Viral Filtration Product containing Ab6 is adjusted with 1.4 M Na₂SO₄, 1 kg Viral Filtered Product to 2 kg 1.4 M Na₂SO₄. Post 1.4 M Na₂SO₄ addition the feed is titrated to a target pH of 7.0 with 1 M Tris base, resulting in the HIC load. The HIC load was filtered through a Millipore SHC 0.5/0.2 μm filter and loaded onto the column. Table 6 details the operating steps and parameters for the HIC chromatography: column equilibration, and HIC chromatography process. The column effluent absorbance was monitored on-line at a wavelength of 280 nm and used to collect the unadjusted HIC product. The unadjusted HIC product was titrated to a target pH of 5.8 with 1 M Acetic Acid solution. Post pH adjustment, 1 kg of HIC Product was diluted with 2 kg 10 mM Histidine, 70 mM Arginine pH 5.8 and filtered through a Millipore SHC 0.5/0.2 μm filter resulting in the Ultrafiltrated Difiltrated (UFDF) load.

TABLE 6
Summary of the HIC Processing Steps

| | | | Buffer | |
|---|---|---|---|---|
| | | | Conductivity | Buffer |
| Step | Buffer | Flow | (mS/cm) | pH |
| Equilibration | 25 mM NaPhosphate, | Down | 95 ± 5.0 | 7.0 ± |
| | 1.13M Na₂SO₄ | | | 0.2 |
| Load | HIC Column Load | Down | 82 ± 4.0 | 7.0 ± |
| | | | | 0.2 |
| Wash | 25 mM NaPhosphate, | Down | 95 ± 5.0 | 7.0 ± |
| | 1.13M Na₂SO₄ | | | 0.2 |
| Elution | 25 mM NaPhosphate, | Down | 63 ± 3.0 | 7.0 ± |
| | 560 mM Na₂SO₄ | | | 0.2 |

The in-process intermediates of the above batch along with the corresponding chromatography strip samples were tested for lipase identification by liquid chromatography-mass spectrometry (LC-MS) (Table 7). PLBL2 and Clusterin were found in the load and strip samples but absent from the HIC elution pool samples.

HCP proteomics by LC-MS/MS (tandem MS data is acquired in data-dependent acquisition or DDA mode) is developed to provide HCP profiling, including HCP identifications and relative quantitation, of bioprocess intermediates and drug substances (DS). Samples including HIC column load solution, HIC column-elution pool, and HIC column-stripped sample were subjected to denaturation, DTT reduction, IAA alkylation, and trypsin digestion. The digested samples were then analyzed by LC-MS/MS (DDA) performed on a Waters H-class UPLC-Thermo QE orbitrap system. Waters ACQUITY UPLC PEPTIDE CSH C18 column (130A, 1.7 μm, 1×150 mm) were used for separation and 0.1% FA in water and 0.1% FA in ACN were used as mobile phase A and B. CHO database was searched by Thermo PD 2.2 for protein identification (mass accuracy ≤10 ppm for MS and ≤0.02 Da for MS/MS; ≤1% FDR; ≥2 unique peptide IDs per protein). Relative quantitation of an HCP is achieved by Σ XIC MS1 peak area(s) of its unique peptides extracted by PD 2.2.

TABLE 7
Relative quantification of endogenous PLBL2
in Ab6 process intermediates

| Sample | PLBL2 (ppm) |
|---|---|
| HIC Load | 147.5 |
| HIC Pool | Not detectable |
| HIC Strip | 1253.0 |

### Example 6: PS-80 Stability Increased as Host Cell Lipases were Removed

Ab6A injection is a sterile, preservative-free solution that requires dilution for intravenous infusion. Ab6A is a fixed dose combination of anti-LAG3 antibody Ab6 and anti-PD-1 antibody MK-3475 (pembrolizumab), each single-use vial contains 40 mg of Ab6 and 10 mg of MK-3475 in a 2.0 mL fill. The drug product composition is 20.0 mg/mL Ab6, 5.0 mg/mL MK-3475, 54 mg/mL sucrose; 0.2 mg/mL polysorbate 80, 10 mM histidine buffer at pH 5.8; 56 mM L-arginine; and 8 mM L-methionine. The Ab6 drug substance from Example 4 was used to formulate the Ab6A drug product.

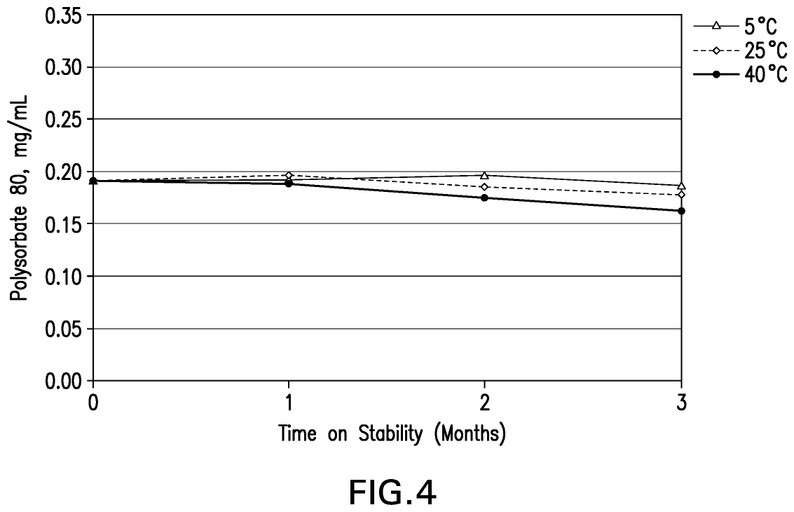

Polysorbate 80 (PS-80) was run for Ab6A drug product up to 3 months on stability (FIG. 4). At 5° C., little change was seen in % PS-80 content at the 3 month time point (0.19 mg/ml). Slight decreases in PS-80 at 25° C. were seen at 3 months (0.18 mg/ml) and a slightly more pronounced decrease was seen at the same interval for the 40° C. condition (0.16 mg/ml).

Polysorbate 80 was determined using a high-performance liquid chromatography (HPLC) with a mixed mode column (Waters Oasis Max column, 2.1×20 mm, 30 m) in combination with a post column switch and Charged Aerosol Detection (CAD). The Corona Calif.D is a mass sensitive detector that responds to essentially all non-volatile and some semi-volatile compounds in the sample which elute from the column. Mobile Phase A: 0.5% (v/v) acetic acid in water and Mobile Phase B: 0.5% (v/v) acetic acid in isopropyl alcohol were used in a gradient setting with flow rate of 1 mL/min. The calculation of the polysorbate 80 concentration is performed with a quadratic fit calibration line on the PS-80 standards and reported as polysorbate 80 concentration (mg/mL) in the sample solutions.

The PS-80 stability was compared between two Ab6 Drug Substance (DS) samples that were generated from a two-column and a three-column purification scheme. The two-column purification scheme included Protein A and AEX. The resulting AEX pool (AEX) was formulated into 25 mg/mL Ab6; 50 mg/mL sucrose; 0.2 mg/mL polysorbate 80; 10 mM histidine buffer at pH 5.8; and 70 mM L-Arginine-HCl. and is referred to as “AEX DS.” The three-column purification scheme included Protein A, AEX, and HIC bind and elute or flowthrough (HIC B&E DS or HIC FT DS). The resulting HIC pool was formulated into 25 mg/mL of the Ab6; 50 mg/mL sucrose; 0.2 mg/mL polysorbate 80; 10 mM L-histidine buffer at pH 5.8; 70 mM L-arginine and 10 mM L-methionine. Vials were placed in the stability chambers at 5° C.±3° C.; 25° C.±3° C., 60%+5% relative humidity (RH). Samples were pulled and tested for PS-80 concentration at 2, 4, 6, 14-week intervals.

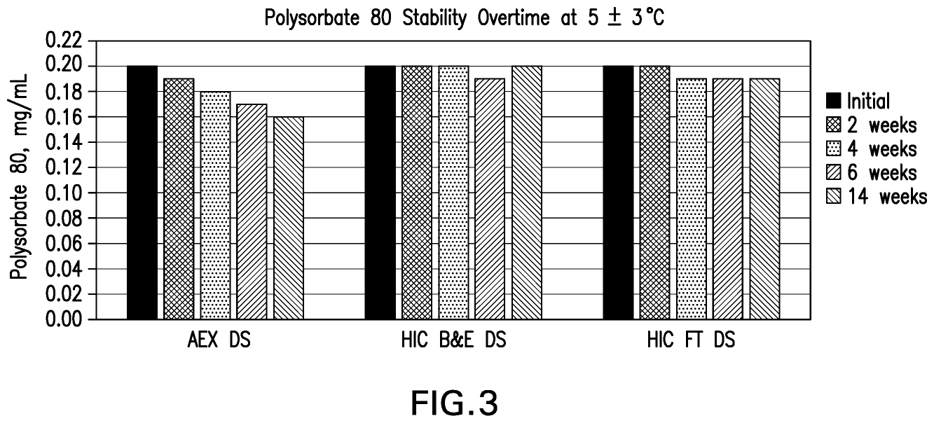

As shown in FIG. 3, the PS-80 concentration in AEX DS decreased from 0.20 (0 week) to about 0.17 mg/mL (6 weeks) at 5° C. The degradation of PS-80 increased as the storage temperature increased. For example, at 25° C., the PS-80 concentration in AEX DS decreased from 0.20 (0 week) to 0.12 mg/mL (6 weeks). On the other hand, the PS-80 concentration in both HIC B&E DS and HIC FT DS did not change significantly over time at both temperatures. The assay variability for the PS-80 stability method is +10%. When evaluating data any drift of <±10% from the initial timepoint reported value can be viewed as being similar in value. It is hypothesized that the presence of PLBL2 in the AEX pool could be one potential cause for the PS-80 concentration decline at 5-25° C. in the AEX DS. Adding a third HIC column can effectively remove lipases and improve the PS-80 stability in the HIC DS.

TABLE 8
Polysorbate 80 Stability Overtime at 5 ± 3° C.

| | Initial | 2 weeks | 4 weeks | 6 weeks | 14 weeks |
|---|---|---|---|---|---|
| Sample ID | Concentration Polysorbate 80, mg/mL | | | | |
| AEX DS | 0.20 | 0.19 | 0.18 | 0.17 | 0.16 |
| HIC B&E | 0.20 | 0.20 | 0.20 | 0.19 | 0.20 |
| DS | | | | | |
| HIC FT DS | 0.20 | 0.20 | 0.19 | 0.19 | 0.19 |

Additional PS80 stability was tested for Ab6 drug substance purified through Example 4 and formulated into 25 mg/mL of the Ab6; 50 mg/mL sucrose; 0.2 mg/mL polysorbate 80; 10 mM L-histidine buffer at pH 5.8; 70 mM L-arginine and 10 mM L-methionine. Vials were placed in the stability chamber at 5° C.+3° C. Samples were pulled and tested for PS-80 concentration at 1, 3, 6, 9 and 12 month intervals (Table 9). The PS-80 concentration did not change significantly over time and was within the assay variability for the PS-80 stability method of 10%.

TABLE 9
Polysorbate 80 Stability Overtime at 5 ± 3° C.

| | | 1 | 3 | 6 | 9 | 12 |
|---|---|---|---|---|---|---|
| | Initial | month | month | month | month | month |
| Sample ID | Concentration Polysorbate 80, mg/mL | | | | | |
| Ab6 Batch 1 | 0.21 | 0.21 | 0.21 | 0.21 | 0.22 | 0.22 |

1. A method of separating a host cell lipase from a composition comprising an anti-LAG3 antibody or antigen-binding fragment and a host cell lipase through a hydrophobic interaction chromatographic (HIC) process, comprising:
(a) passing a load fluid comprising the composition through the HIC resin under a loading operating condition; and
(b) collecting the anti-LAG3 antibody or antigen-binding fragment in a flowthrough;
wherein separation factor (α) is the ratio of the partition coefficient (Kₚ) for the lipase to the Kₚ for the anti-LAG3 antibody or antigen-binding fragment, and wherein log α is larger than 0.5 under the loading operating condition; wherein the anti-LAG3 antibody or antigen binding fragment comprises: (a) light chain CDRs of SEQ ID NOs: 6, 7 and 8 and (b) heavy chain CDRs of SEQ ID NOs: 9, 10 and 11.

2. A method of separating a host cell lipase from a composition comprising an anti-LAG3 antibody or antigen-binding fragment and a host cell lipase through a hydrophobic interaction chromatographic (HIC) process, comprising:
(a) passing a load fluid comprising the composition through the HIC resin under a loading operating condition; and
(b) eluting the anti-LAG3 antibody or antigen-binding fragment from the chromatographic resin with an elution solution under an elution operating condition;
wherein separation factor (α) is the ratio of the partition coefficient (Kₚ) for the lipase to the Kₚ for the anti-LAG3 antibody or antigen-binding fragment, and wherein log α is larger than 0.5 under the elution operating condition; wherein the anti-LAG3 antibody or antigen binding fragment comprises: (a) light chain CDRs of SEQ ID NOs: 6, 7 and 8 and (b) heavy chain CDRs of SEQ ID NOs: 9, 10 and 11.

3. A method of separating a host cell lipase from a composition comprising an anti-LAG3 antibody or antigen-binding fragment and a host cell lipase through a Cation Exchange (CEX) process, comprising:
(a) passing a load fluid comprising the composition through the CEX resin under a loading operating condition; and
(b) eluting the anti-LAG3 antibody or antigen-binding fragment from the chromatographic resin with an elution solution under an elution operating condition;
wherein separation factor (α) is the ratio of the partition coefficient (Kₚ) for the lipase to the Kₚ for the anti-LAG3 antibody or antigen-binding fragment, and wherein log α is larger than 0.5 under the elution operating condition; wherein the anti-LAG3 antibody or antigen binding fragment comprises: (a) light chain CDRs of SEQ ID NOs: 6, 7 and 8 and (b) heavy chain CDRs of SEQ ID NOs: 9, 10 and 11.

4. A method of improving polysorbate-80 (PS-80) stability in an anti-LAG3 antibody or antigen-binding fragment formulation, comprising:
(a) passing a load fluid comprising a host cell lipase and the anti-LAG3 antibody or antigen-binding fragment through a HIC chromatographic resin under a loading operating condition;
(b) collecting the anti-LAG3 antibody or antigen-binding fragment in a flowthrough; and
(c) formulating the anti-LAG3 antibody or antigen-binding fragment so that the anti-LAG3 antibody or antigen-binding fragment formulation is a PS-80-containing solution;
wherein separation factor (α) is the ratio of the partition coefficient (Kₚ) for the lipase to the Kₚ for the anti-LAG3 antibody or antigen-binding fragment, and wherein log α is larger than 0.5 under the loading operating condition; wherein the anti-LAG3 antibody or antigen binding fragment comprises: (a) light chain CDRs of SEQ ID NOs: 6, 7 and 8 and (b) heavy chain CDRs of SEQ ID NOs: 9, 10 and 11.

5-15. (canceled)

16. A method of separating a PLBL2 or LPLA2 from a composition comprising an anti-LAG3 antibody or antigen-binding fragment and a PLBL2 or LPLA2 through a hydrophobic interaction chromatographic process, comprising:
(a) passing a load fluid comprising the composition through a hydrophobic interaction chromatographic resin; and
(b) collecting the anti-LAG3 antibody or antigen-binding fragment in a flowthrough;
and wherein the load fluid has a conductivity of about 25 to 80 mS/cm; wherein the anti-LAG3 antibody or antigen binding fragment comprises: (a) light chain CDRs of SEQ ID NOs: 6, 7 and 8 and (b) heavy chain CDRs of SEQ ID NOs: 9, 10 and 11.

17. A method of separating a PLBL2 or LPLA2 from a composition comprising an anti-LAG3 antibody or antigen-binding fragment and a PLBL2 or LPLA2 through a hydrophobic interaction chromatographic process, comprising:
(a) passing a load fluid comprising the composition through a HIC resin; and
(b) eluting the anti-LAG3 antibody or antigen-binding fragment from the HIC resin with an elution solution; wherein the elution solution has a conductivity of about 25 to 80 mS/cm;
wherein the anti-LAG3 antibody or antigen binding fragment comprises: (a) light chain CDRs of SEQ ID NOs: 6, 7 and 8 and (b) heavy chain CDRs of SEQ ID NOs: 9, 10 and 11.

18-22. (canceled)

23. A composition comprising an anti-LAG3 antibody or antigen-binding fragment and less than 2 ppm of a host cell lipase, wherein the anti-LAG3 antibody or antigen binding fragment comprises: (a) light chain CDRs of SEQ ID NOs: 6, 7 and 8 and (b) heavy chain CDRs of SEQ ID NOs: 9, 10 and 11.

24. The composition of claim 23, comprising less than 1 ppm of a host cell lipase.

25. The composition of claim 23, wherein the lipase is selected from the group consisting of PLBL2, LPL, LPLA2, LP-PLA2, and LAL.

26. The composition of claim 23, wherein the lipase is PLBL2.

27. The composition of claim 23, wherein the level of the host cell lipase is measured by liquid chromatography-mass spectrometry (LC-MS) or liquid chromatography-multiple reaction (LC-MRM-MS).

28. The composition of claim 23, wherein the composition is obtainable by a HIC process comprising the steps of:
(a) passing a load fluid comprising a composition comprising the anti-LAG3 antibody or antigen-binding fragment and host cell lipase through a HIC resin under a loading operating condition; and
(b) eluting the anti-LAG3 antibody or antigen-binding fragment thereof with an elution solution with a pH from about 5 to about 7.5, and a conductivity of about 25-80 mS/cm; or
(c) collecting the anti-LAG3 antibody or antigen-binding fragment thereof in the flowthrough using loading operation conditions with a pH from about 5 to about 7.5, and a conductivity of about 25-80 mS/cm.

29. The composition of claim 23, wherein the composition is obtainable by a HIC process comprising the steps of:
(a) passing a load fluid comprising a composition comprising the anti-LAG3 antibody or antigen-binding fragment and host cell lipase through a HIC resin under a loading operating condition; and
(b) eluting the anti-LAG3 antibody or antigen-binding fragment thereof with an elution solution with a pH from about 5 to about 7.5, and a conductivity of about 50-70 mS/cm; or
(c) collecting the anti-LAG3 antibody or antigen-binding fragment thereof in the flowthrough using loading operation conditions with a pH from about 5 to about 7.5, and a conductivity of about 50-70 mS/cm.

30. The composition of claim 28, wherein the HIC chromatography is preceded by Protein A chromatography operated in bind and elute mode and an AEX chromatography operated in a flowthrough mode.

31. A pharmaceutical composition comprising an anti-LAG3 antibody or antigen-binding fragment and polysorbate 80 (PS80) or polysorbate 20 (PS20), wherein at 1 month, 3 months, 6 months, 9 months or 12 months at 2-8° C., the concentration of PS80 or PS20 is maintained at ≥90% of the concentration when formulated, wherein the anti-LAG3 antibody or antigen binding fragment comprises: (a) light chain CDRs of SEQ ID NOs: 6, 7 and 8 and (b) heavy chain CDRs of SEQ ID NOs: 9, 10 and 11.

32. The pharmaceutical composition of claim 31 that comprises about 0.2 mg/mL polysorbate 80 when formulated.

33. The pharmaceutical composition of claim 31 that comprises about 20.0 mg/mL of the anti-LAG3 antibody or antigen-binding fragment, about 5.0 mg/mL pembrolizumab, about 54 mg/mL sucrose; about 0.2 mg/mL polysorbate 80, about 10 mM histidine buffer at pH about 5.8; about 56 mM L-arginine; and about 8 mM L-methionine when formulated.

34. The pharmaceutical composition of claim 31 that comprises about 25.0 mg/mL of the anti-LAG3 antibody or antigen-binding fragment; about 50 mg/mL sucrose; about 0.2 mg/mL polysorbate 80; about 10 mM histidine buffer at pH about 5.8; about 70 mM L-Arginine-HCl; and optionally about 10 mM L-methionine.

35. The method, composition or pharmaceutical composition of claim 1, wherein the anti-LAG3 antibody or antigen binding fragment comprises a heavy chain variable region comprising SEQ ID NO:5 and the light chain comprises a light chain variable region comprising SEQ ID NO: 4.

36. The method, composition or pharmaceutical composition of claim 1, wherein the anti-LAG3 antibody comprises a heavy chain and a light chain, and wherein the heavy chain comprises SEQ ID NO:3 and the light chain comprises SEQ ID NO:2.

37. The method, composition or pharmaceutical composition of claim 36, wherein the anti-LAG3 antibody has up to three consecutive amino acid substitutions in the light chain framework regions or constant region and six, five, four, three, two or one conservative amino acid substitutions is the heavy chain framework regions or the constant region, and optionally has a deletion of the C-terminal lysine residue of the heavy chain.
